(19) United States
(12) Patent Application Publication
(10) Pub. No.: US 2022/0096461 A1
(43) Pub. Date: Mar. 31, 2022

(54) LIQUID ORAL DOSAGE FORMULATIONS OF METHYLNALTREXONE

(71) Applicant: Bausch Health Ireland Limited, Dublin (IE)

(72) Inventors: Brian R. Rohrs, Fairport, NY (US); Daniel J. Stein, Rochester, NY (US); Vijay Kumar, Webster, NY (US); Eric Phillips, Ontario, NY (US)

(73) Assignee: Bausch Health Ireland Limited, Dublin (IE)

(21) Appl. No.: 17/495,413

(22) Filed: Oct. 6, 2021

Related U.S. Application Data
(63) Continuation of application No. PCT/EP2020/062794, filed on May 7, 2020.
(60) Provisional application No. 63/010,528, filed on Apr. 15, 2020.
(60) Provisional application No. 62/844,613, filed on May 7, 2019.

(51) Int. Cl. A61K 31/485; A61K 47/44

(52) U.S. Cl. CPC A61K 31/485; A61K 47/44

(57) ABSTRACT

Provided herein are pharmaceutical compositions comprising methylnaltrexone, lauryl sulfate or docusate, and at least one of an oil, a surfactant or a cosolvent, wherein the methylnaltrexone and lauryl sulfate or docusate are present as an ion pair. The pharmaceutical compositions may further comprise a surfactant so as to provide self-emulsifying methylnaltrexone delivery systems.

### CROSS-REFERENCE TO RELATED APPLICATIONS

This application is a continuation of International Application No. PCT/EP2020/062794, filed on May 7, 2020, which claims the benefit of U.S. Provisional Application No. 62/844,613, filed on May 7, 2019 and U.S. Provisional Application No. 63/010,528, filed on Apr. 15, 2020, the entire contents of which are expressly incorporated herein by reference.

### BACKGROUND

Opioids are widely used to treat patients with pain. Opioids are narcotic medications that activate opioid receptors located in the central nervous system to relieve pain. Opioids, however, also react with receptors outside of the central nervous system, resulting in side effects including constipation, nausea, vomiting, urinary retention, and severe itching. Notable are the effects of opioids in the gastrointestinal (GI) tract where these drugs inhibit gastric emptying and peristalsis in the intestines, thereby decreasing the rate of intestinal transit and producing constipation. The use of opioids in treating pain is often limited due to these undesired side effects, which can be debilitating and often cause patients to refuse the use of opioid analgesics. Accordingly, new therapies and formulations are desired in the field to manage such undesired side effects.

### SUMMARY

Opioid receptor antagonists, such as naloxone, naltrexone, and nalmefene, have been studied as a means of antagonizing the undesirable peripheral side effects of opioids. However, these agents not only act on peripheral opioid receptors but also on opioid receptors in the central nervous system, sometimes reversing the beneficial and desired analgesic effects of opioids or causing symptoms of opioid withdrawal. Preferable approaches for use in controlling opioid-induced side effects include administration of peripheral acting opioid receptor antagonists that do not readily cross the blood-brain barrier.

The peripheral μ opioid receptor antagonist methylnaltrexone has been studied since the late 1970s and has been used in patients to reduce opioid-induced side effects such as constipation, pruritus, nausea, and urinary retention (see, e.g., U.S. Pat. Nos. 5,972,954, 5,102,887, 4,861,781, and 4,719,215; and Yuan et al., Drug and Alcohol Dependence 1998, 52, 161). The dosage form of methylnaltrexone used most often in these studies has been a solution of methylnaltrexone for intravenous injection. See also U.S. Pat. No. 6,559,158. Subcutaneous methylnaltrexone formulations, marketed under the brand name RELISTOR®, are approved for the treatment of opioid-induced constipation in adults with chronic non-cancer pain and in adults with advanced illness who are receiving palliative care. For example, in clinical studies, 59% of patients with chronic non-cancer pain who received a 12 mg subcutaneous injection of methylnaltrexone to treat opioid-induced constipation had three or more spontaneous bowel movements each week for four weeks. However, it has been challenging to prepare oral dosage forms of methylnaltrexone. See e.g., U.S. Pat. Nos. 6,419,959, 6,274,591, 6,559,158.

Although, oral RELISTOR® tablets have proven to be a safe and effective treatment for opioid-induced constipation, there is a desire to reduce the 450 mg methylnaltrexone dose, which is administered as three 150 mg tablets. Additionally, or alternatively, because laxation following subcutaneous injection has been correlated with higher Cₘₐₓ than the Cₘₐₓ observed following oral administration, development of oral dosage forms that result in greater systemic exposure is desired. At the same time, decreasing Tₘₐₓ to achieve a faster laxation response is also desired.

Turning to particular aspects of the invention described herein, a pharmaceutical composition in a liquid oral dosage form is disclosed that includes: (a) an ion pair having the formula:

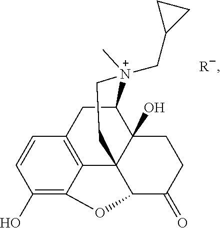

wherein R′ may be an anion, and (b) an oil, surfactant, cosolvent, or combination thereof. In some embodiments, R′ may be any anion that allows for the formation of the ion pair. In some embodiments, R′ may be an anion selected from the group consisting of lauryl sulfate and docusate.

In a particular embodiment, the anion may be lauryl sulfate. Alternatively, the anion may be docusate.

In some embodiments, the pharmaceutical composition comprises an oil and a surfactant. In some embodiments, the pharmaceutical composition comprises an oil and at least two surfactants. In some embodiments, the pharmaceutical composition comprises a surfactant and a cosolvent. In some embodiments, the pharmaceutical composition comprises at least two surfactants. In some embodiments, the pharmaceutical composition contains no oil, but comprises a surfactant. In some embodiments, the pharmaceutical composition contains no oil, but at least two surfactants.

In certain embodiments, the pharmaceutical composition includes about 1% to about 75%, about 10% to about 60%, about 15% to about 50%, or about 20% to about 40% by weight, of the ion pair.

In another embodiment, the oil may be at least one of glyceryl monooleate, glyceryl monolinoleate, propylene glycol dicaprolate/dicaprate, soybean oil, polyglyceryl-3 dioleate, oleic acid, glyceryl caprylate, medium chain triglycerides, and a combination thereof. In some embodiments, the oil may be glyceryl monolinoleate. In some embodiments, the oil may be oleic acid. In other embodiments, the oil may be glyceryl caprylate. In yet further embodiments, the oil includes medium chain triglycerides.

In a further embodiment, the pharmaceutical composition includes at least two, three, four, five, or more oils. In some embodiments, the pharmaceutical composition includes at least two oils. For example, in some embodiments, the oil includes glyceryl caprylate and medium chain triglycerides. In alternative embodiments, the pharmaceutical composition includes at least three oils, for example, caprylic/capric/succinic triglyceride, glyceryl caprylate (mono- and diglycerides), and oleic acid. In various embodiments, the total oil content of the pharmaceutical composition is about 10% to about 80%, about 10% to about 20%, about 20% to about 50%, or about 50% to about 70% by weight. In some embodiments, the pharmaceutical composition has no oil.

In certain embodiments, the pharmaceutical composition described herein further includes a surfactant. Suitable surfactants for use in pharmaceutical compositions described herein include, but are not limited to, oleoyl polyoxyl-6 glycerides, linoleoyl polyoxyl-6 glycerides, caprylocaproyl polyoxyl-8 glycerides, polysorbate 80, polyoxyl 40 hydrogenated castor oil, polyoxyl 15 hydroxystearate, lauroyl polyoxyl-32 glycerides, and/or a combination thereof. In one embodiment, the surfactant includes caprylocaproyl polyoxyl-8 glycerides. In another embodiment, the surfactant includes polysorbate 80. In yet another embodiment, the surfactant includes linoleoyl polyoxyl-6 glycerides. In yet another embodiment, the surfactant includes polyoxyl 40 hydrogenated castor oil. In yet another embodiment, the surfactant includes polyoxyl 15 hydroxystearate. In yet another embodiment, the surfactant includes lauroyl polyoxyl-32 glycerides.

In various embodiments, pharmaceutical compositions include about 10% to about 70%, about 15% to about 40%, or about 20% to about 35% of the surfactant by weight.

In some embodiments, the pharmaceutical composition includes one or more cosurfactants. In exemplary embodiments, the pharmaceutical composition includes up to about 20% (w/w) cosurfactant. For example, the cosurfactant may include Capryol™ (propylene glycol caprylate) and/or Lauroglycol™ (propylene glycol monolaurate).

In certain embodiments, the pharmaceutical compositions include about 1 mg to about 100 mg, about 50 mg to about 900 mg, about 75 mg to about 850 mg, about 100 mg to about 850 mg, about 150 mg to about 850 mg, about 200 mg to about 800 mg, or about 200 mg to about 700 mg of the ion pair. In other embodiments, the pharmaceutical compositions of the disclosure include about 1 mg, about 10 mg, about 25 mg, about 50 mg, about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg or about 900 mg of the ion pair.

In further embodiments, the pharmaceutical composition includes about 1 mg to about 100 mg, about 50 mg to about 800 mg, about 100 mg to about 750 mg, about 150 mg to about 750 mg, or about 200 mg to about 700 mg of the methylnaltrexone and lauryl sulfate. In alternative embodiments, the pharmaceutical composition includes about 1 mg to about 100 mg, about 50 mg to about 900 mg, about 100 mg to about 900 mg, about 150 mg to about 850 mg, or about 200 mg to about 800 mg of the methylnaltrexone and docusate.

In further embodiments, the pharmaceutical compositions include water, such that the liquid composition is an emulsion. In another alternative embodiment, the pharmaceutical composition forms an emulsion upon contact with aqueous liquids, e.g., gastric and/or intestinal juices.

In some embodiments, the cosolvent may be one or more of triacetin, ethanol, glycerol, propylene glycol, and polyethylene glycol (e.g., PEG-400). In some embodiments, the cosolvent comprises ethanol.

In another aspect, a pharmaceutical composition in a liquid oral dosage form is described herein that includes: (a) an ion pair having the formula:

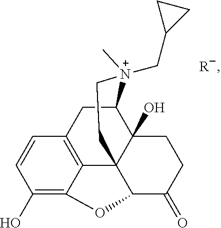

wherein R′ may be an anion selected from the group consisting of lauryl sulfate and docusate. In one embodiment, the anion may be lauryl sulfate. In yet another embodiment, the anion may be docusate.

In a further aspect, a pharmaceutical composition in a liquid oral dosage form includes (i) methylnaltrexone, (ii) lauryl sulfate or docusate and (iii) one or more of an oil, surfactant, and a cosolvent, wherein the methylnaltrexone and lauryl sulfate or docusate are present in substantially equal molar amounts. As used herein, the term “substantially equal molar” means the moles of lauryl sulfate or docusate are within 5%, 4%, 3%, 2%, 1%, 0.1%, or 0.01% of the moles of methylnaltrexone. In one embodiment, the pharmaceutical composition includes lauryl sulfate. In another embodiment, the pharmaceutical composition includes docusate.

In one embodiment, the pharmaceutical composition includes methylnaltrexone and lauryl sulfate in an amount that is about 1% to about 75%, about 10% to about 60%, about 15% to about 50%, or about 20% to about 40% of the pharmaceutical composition by weight. In another embodiment, the pharmaceutical composition includes methylnaltrexone and docusate in an amount that is about 1% to about 75%, about 10% to about 60%, about 15% to about 50%, or about 20% to about 40% of the pharmaceutical composition by weight.

In some embodiments, the oil includes at least one of glyceryl monooleate, glyceryl monolinoleate, propylene glycol dicaprolate/dicaprate, soybean oil, polyglyceryl-3 dioleate, oleic acid, glyceryl caprylate, medium chain triglycerides, and a combination thereof. For example, in one embodiment, the oil includes glyceryl monolinoleate. In another exemplary embodiment, the oil includes oleic acid. In a further embodiment, the oil includes glyceryl caprylate. In a particular embodiment, the oil includes medium chain triglycerides. In a certain embodiment, the oil includes at least two oils, e.g., glyceryl caprylate and medium chain triglycerides. In some embodiments, the total oil content of the pharmaceutical composition is about 10% to about 80%, about 10% to about 20%, about 20% to about 50%, or about 50% to about 70% by weight of the composition.

In further embodiments, the pharmaceutical composition includes a surfactant. Suitable surfactants for use in the pharmaceutical compositions of the disclosure include oleoyl polyoxyl-6 glycerides, linoleoyl polyoxyl-6 glycerides, caprylocaproyl polyoxyl-8 glycerides, polysorbate 80, polyoxyl 40 hydrogenated castor oil, polyoxyl 15 hydroxystearate, lauroyl polyoxyl-32 glycerides, or a combination thereof. In an exemplary embodiment, the surfactant includes caprylocaproyl polyoxyl-8 glycerides. In another exemplary embodiment, the surfactant is polysorbate 80. In yet another exemplary embodiment, the surfactant is linoleoyl polyoxyl-6 glycerides. In some embodiments, the surfactant includes polyoxyl 40 hydrogenated castor oil. In some embodiments, the surfactant includes polyoxyl 15 hydroxystearate. In some embodiments, the surfactant includes lauroyl polyoxyl-32 glycerides. In some embodiments, the pharmaceutical composition includes about 10% to about 70%, about 15% to about 40%, or about 20% to about 35% of the surfactant by weight of the composition.

The pharmaceutical composition may include about 1 mg to about 100 mg, about 50 mg to about 800 mg, about 100 mg to about 750 mg, about 150 mg to about 750 mg, or about 200 mg to about 700 mg of the methylnaltrexone and lauryl sulfate. For example, the pharmaceutical composition may include about 1 mg, about 10 mg, about 25 mg, about 50 mg, about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg or about 700 mg of the methylnaltrexone and lauryl sulfate.

Alternatively, the pharmaceutical composition may include about 1 mg to about 100 mg, about 50 mg to about 900 mg, about 100 mg to about 900 mg, about 150 mg to about 850 mg, or about 200 mg to about 800 mg of the methylnaltrexone and docusate. In exemplary pharmaceutical compositions, methylnaltrexone and docusate are present in the pharmaceutical composition in amounts of about 1 mg, about 10 mg, about 25 mg, about 50 mg, about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg about 700 mg, about 750 mg, about 800 mg, about 850 mg or about 900 mg.

In certain embodiments, the pharmaceutical compositions also include water, and the liquid composition is an emulsion. In other embodiments, the composition forms an emulsion upon contact with aqueous liquids.

In another aspect, the invention provides a salt having the formula:

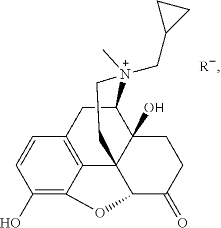

wherein R′ is docusate. In a certain embodiment, the methylnaltrexone and docusate salt may be present in a pharmaceutical composition, for example, as a liquid composition for oral administration. In some embodiments, the pharmaceutical composition further includes one or more of an oil, a surfactant, and a cosolvent. In some embodiments, the pharmaceutical composition includes a surfactant and a cosolvent.

In some embodiments, pharmaceutical compositions of any of the foregoing aspects of the invention are formulated as a capsule, e.g., soft gel capsule, hard gel capsule, or enteric capsule.

In other aspects, methods of treating opioid-induced constipation in a subject in need thereof are provided that include orally administering a pharmaceutical composition as described herein. In certain embodiments, oral administration of the pharmaceutical composition to the subject results in a Cₘₐₓ ranging from about 50 ng/mL to about 200 ng/mL. In certain embodiments, oral administration of the pharmaceutical composition to the subject results in a Tₘₐₓ that is less than about 4 hours, less than about 2 hours, less than about 1 hour, less than about 30 minutes, less than about 15 minutes, or less than about 10 minutes.

### BRIEF DESCRIPTION OF THE DRAWINGS

FIG. 1 shows the average plasma concentration of methylnaltrexone v. time following administration of four oral formulations according to Examples 2.1 to 2.4 and a RELISTOR® tablet (control).

FIG. 2 shows the plasma concentration of methylnaltrexone v. time after administration of a RELISTOR® tablet (control) according to the procedure provided in Example 3.

FIG. 3 shows the average plasma concentration of methylnaltrexone v. time after administration of a self-emulsifying drug delivery system comprising methylnaltrexone-lauryl sulfate according to Example 2.1.

FIG. 4 shows the average plasma concentration of methylnaltrexone v. time after administration of an oil-based liquid formulation comprising methylnaltrexone-docusate according to Example 2.2.

FIG. 5 shows the average plasma concentration of methylnaltrexone v. time after administration of a self-emulsifying drug delivery system comprising methylnaltrexone-docusate in enteric capsules according to Example 2.3.

FIG. 6 shows the average plasma concentration of methylnaltrexone v. time after administration of a self-emulsifying drug delivery system comprising methylnaltrexone-docusate. according to Example 2.4.

### DETAILED DESCRIPTION

The invention described herein is based, at least in part, on the discovery of oral formulations of methylnaltrexone having improved pharmacokinetic properties and response times as compared to prior oral formulations. Specifically, the formulations disclosed herein provide enhanced absorption rates, enhanced Cₘₐₓ and/or reduced Tₘₐₓ, thereby resulting in an improved profile for treating peripheral side effects of opioids, such as constipation.

Specifically, provided herein are pharmaceutical compositions in a liquid oral dosage form including: (a) an ion pair having the formula:

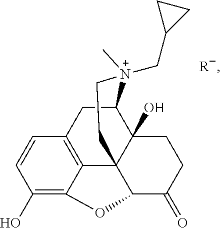

wherein R′ may be an anion selected to provide the ion pair; and (b) one or more of an oil, surfactant, or a cosolvent. In some embodiments, R′ may be an anion selected from the group consisting of lauryl sulfate and docusate In particular embodiments, the liquid oral dosage form includes an oil and a surfactant. In some embodiments, the liquid oral dosage form includes a surfactant and a cosolvent.

In particular embodiments, the pharmaceutical compositions of the invention are formulated and administered as a capsule, e.g., soft gel capsule, hard gel capsule, and enteric gel capsule. In a particular embodiment, the pharmaceutical compositions are formulated as a soft gel capsule.

However, the compositions may alternatively be formulated as tablets. In some embodiments, solid granules can be produced by melt granulation. In other embodiments, waxy powders can be produced by solvent evaporation. In further embodiments, solid granules and/or powders can be produced by spray drying. Such granules and powders can be compressed into tablets in accordance with the inventive subject matter. In some embodiments, lipids are adsorbed onto a solid carrier, such as silicon dioxide, calcium silicate, and/or magnesium aluminometasilicate, which is compressed to make tablets.

### 1. Compositions

### 1.1. Methylnaltrexone Ion Pairs

As used herein, methylnaltrexone refers to (R)—N-methylnaltrexone. (R)—N-methylnaltrexone, a peripherally acting μ opioid receptor antagonist, has been studied and used to treat bowel dysfunction in patients being administered opioids.

Methylnaltrexone is a quaternary amine and, as such, has a positive charge. This charge results in slower absorption rates (as compared to neutral molecules) across membranes. The existing RELISTOR® tablet, as described, for example, in U.S. Pat. No. 9,314,461, combines methylnaltrexone bromide with sodium lauryl sulfate, and relies on in situ formation of the neutral methylnaltrexone and lauryl sulfate ion pair to enhance absorption. The invention described herein, however, is predicated, at least in part, on the finding that the formulation and administration of a pre-existing ion pair of methylnaltrexone and either docusate or lauryl sulfate, can serve to enhance the absorption rate. Because methylnaltrexone has been reported to have a high first-pass metabolism, an increase in the rate of absorption can serve to saturate the metabolic pathway, thereby further enhancing the amount of methylnaltrexone absorbed by the body.

Accordingly, the pharmaceutical compositions of the invention described herein include an ion pair of methylnaltrexone with either lauryl sulfate or docusate, which results in improved pharmacokinetic properties. In a particular embodiment, the pharmaceutical composition includes an ion pair of methylnaltrexone and lauryl sulfate. In an alternative embodiment, the pharmaceutical composition includes an ion pair of methylnaltrexone and docusate.

Ion pairs, generally, are salts that include a hydrophilic active agent (cation or anion) and a more lipophilic pharmaceutically acceptable counterion. See e.g., Krisztina Tkács-Novák & György Száz, Ion-Pair Partition of Quaternary Ammonium Drugs: The Influence of Counter Ions of Different Lipophilicity, Size, and Flexibility, 16(10) PHARMACEUTICAL RESEARCH 1633-38 (1999). Ion pairs may have a greater hydrophobicity than the active agent as measured by a partition coefficient, e.g.,

$$log ( P \frac{octanol}{water} ) .$$

Increasing the lipophilicity of methylnaltrexone through the pre-formation of ion pairs with lauryl sulfate and/or docusate, as described herein, can improve the ability of methylnaltrexone to penetrate membrane barriers and thereby enhance bioavailability and/or efficacy of methylnaltrexone oral formulations.

In some embodiments, the pharmaceutical composition includes about 1% to about 75%, about 10% to about 60%, about 15% to about 50%, or about 20% to about 40% by weight, of the ion pair. In some embodiments, the pharmaceutical composition includes at least 1%, or at least 2%, or at least 3%, or at least 4%, or at least 5%, or at least 6%, or at least 7%, or at least 8%, or at least 9%, or at least 10%, or at least 11%, or at least 12%, or at least 13%, or at least 14%, or at least 15%, or at least 16%, or at least 17%, or at least 18%, or at least 19%, or at least 20%, or at least 21%, or at least 22%, or at least 23%, or at least 24%, or at least 25%, or at least 26%, or at least 27%, or at least 28%, or at least 29%, or at least 30%, or at least 31%, or at least 32%, or at least 33%, or at least 34%, or at least, 35%, or at least 36%, or at least 37%, or at least 38%, or at least 39%, or at least 40%, or at least 41%, or at least 42%, or at least 43%, or at least 44%, or at least 45%, or at least 46%, or at least 47%, or at least 48%, or at least 49%, or at least 50%, or at least 51%, or at least 52%, or at least 53%, or at least 54%, or at least 55%, or at least 56%, or at least 57%, or at least 58%, or at least 59%, or at least 60% by weight, of the ion pair. In some embodiments, the pharmaceutical composition includes at most 1%, or at most 2%, or at most 3%, or at most 4%, or at most 5%, or at most 6%, or at most 7%, or at most 8%, or at most 9%, or at most 10%, or at most 11%, or at most 12%, or at most 13%, or at most 14%, or at most 15%, or at most 16%, or at most 17%, or at most 18%, or at most 19%, or at most 20%, or at most 21%, or at most 22%, or at most 23%, or at most 24%, or at most 25%, or at most 26%, or at most 27%, or at most 28%, or at most 29%, or at most 30%, or at most 31%, or at most 32%, or at most 33%, or at most 34%, or at most, 35%, or at most 36%, or at most 37%, or at most 38%, or at most 39%, or at most 40%, or at most 41%, or at most 42%, or at most 43%, or at most 44%, or at most 45%, or at most 46%, or at most 47%, or at most 48%, or at most 49%, or at most 50%, or at most 51%, or at most 52%, or at most 53%, or at most 54%, or at most 55%, or at most 56%, or at most 57%, or at most 58%, or at most 59%, or at most 60% by weight, of the ion pair. In some embodiments, the pharmaceutical composition includes about 1%, or about 2%, or about 3%, or about 4%, or about 5%, or about 6%, or about 7%, or about 8%, or about 9%, or about 10%, or about 11%, or about 12%, or about 13%, or about 14%, or about 15%, or about 16%, or about 17%, or about 18%, or about 19%, or about 20%, or about 21%, or about 22%, or about 23%, or about 24%, or about 25%, or about 26%, or about 27%, or about 28%, or about 29%, or about 30%, or about 31%, or about 32%, or about 33%, or about 34%, or about 35%, or about 36%, or about 37%, or about 38%, or about 39%, or about 40%, or about 41%, or about 42%, or about 43%, or about 44%, or about 45%, or about 46%, or about 47%, or about 48%, or about 49%, or about 50%, or about 51%, or about 52%, or about 53%, or about 54%, or about 55%, or about 56%, or about 57%, or about 58%, or about 59%, or about 60% by weight, of the ion pair.

In certain embodiments, where the pharmaceutical composition includes an ion pair of methylnaltrexone and lauryl sulfate, the ion pair is present at about 1% to about 50%, about 5% to about 45%, about 10% to about 40%, about 10% to about 35%, about 10% to about 30%, or about 15% to about 25% by weight of the composition. For example, the methylnaltrexone and lauryl sulfate ion pair may be present in an amount of at least about 10%, 11%, 12%, 13%, 14%, 15%, 16%, 17%, 18%, 19%, 20%, 21%, 22%, 23%, 24%, 25%, 26%, 27%, 28%, 29% or 30% by weight of the composition. In some embodiments, the methylnaltrexone and lauryl sulfate ion pair may be present in an amount of at most about 10%, 11%, 12%, 13%, 14%, 15%, 16%, 17%, 18%, 19%, 20%, 21%, 22%, 23%, 24%, 25%, 26%, 27%, 28%, 29% or 30% by weight of the composition. In some embodiments, the methylnaltrexone and lauryl sulfate ion pair may be present in an amount of about 10%, 11%, 12%, 13%, 14%, 15%, 16%, 17%, 18%, 19%, 20%, 21%, 22%, 23%, 24%, 25%, 26%, 27%, 28%, 29% or 30% by weight of the composition.

In certain embodiments, where the pharmaceutical composition includes an ion pair of methylnaltrexone and docusate, the ion pair is present at about 1% to about 50%, about 10% to about 50%, about 15% to about 45%, about 20% to about 40%, about 15% to about 30%, or about 30% to about 45% by weight of the composition. For example, the methylnaltrexone and docusate ion pair may be present in an amount of at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44% or 45% by weight of the composition. In some embodiments, the methylnaltrexone and docusate ion pair may be present in an amount of at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44% or 45% by weight of the composition. In some embodiments, the methylnaltrexone and docusate ion pair may be present in an amount of about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44% or 45% by weight of the composition.

In some embodiments, the pharmaceutical composition includes about 1 mg to about 100 mg, about 50 mg to about 900 mg, about 75 mg to about 850 mg, about 100 mg to about 850 mg, about 150 mg to about 850 mg, about 200 mg to about 800 mg, or about 200 mg to about 700 mg of the ion pair. In some embodiments, the pharmaceutical composition includes about 1 mg, about 10 mg, about 25 mg, about 50 mg, about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg or about 900 mg of the ion pair. In some embodiments, the pharmaceutical composition includes at least about 1 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, or 900 mg of the ion pair. In some embodiments, the pharmaceutical composition includes at most about 1 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, or 900 mg of the ion pair.

### 1.2. Lipid-Based Drug Delivery Systems

The pharmaceutical compositions as disclosed herein are lipid-based drug delivery systems. Lipid-based drug delivery systems employ oils and/or surfactants to promote oral drug absorption by stimulating bile flow and pancreatic juice secretion; prolonging gastric emptying; increasing membrane fluidity; opening tight junctions, which facilitates drug permeability through the intestinal epithelium; stimulating chylomicron secretion; inhibiting efflux transporters; enhancing drug uptake through the lymphatic pathway, thus bypassing initial metabolism by the liver. Medium chain lipids (C_{<12}) diffuse across the enterocyte directly into blood vessels. Long unsaturated chain lipids (C_{18:1}, C_{18:2}) are absorbed via the lymphatic pathway. See e.g., Sandeep Kalepu et al., Oral lipid-based drug delivery systems an overview, 3(6) ACTA PHARMACEUTICA SINICA B 361-72 (2013).

As used herein, the term “oil” refers to pharmaceutically acceptable lipids having unsaturated fatty acid chains that are liquid at room temperature. Oils include mono-, di-, and triglycerides as well as fatty acids. Long-, medium-, and short-chain glycerides are suitable for use in the pharmaceutical compositions disclosed herein.

As used herein, the term “surfactant” refers to any amphipathic compounds (molecules or ions) that include hydrophilic and lipophilic moieties. Surfactants often operate by accumulating at oil-water interfaces, such that the hydrophilic part is oriented towards the water phase and the lipophilic part towards the hydrophobic phase, thereby reducing surface tension. Suitable surfactants include water-insoluble surfactants, water-dispersible surfactants, and water-soluble surfactants. It should be appreciated that surfactants employed in the disclosed pharmaceutical compositions are present at pharmaceutically acceptable concentrations. However, as used herein, the term “surfactant” or “cosurfactant” excludes sodium alkyl sulfates such as sodium lauryl sulfate.

Surfactants and oils can further be characterized by their hydrophobic-lipophilic balance (“HLB”) values, which is the balance of the size and strength of the hydrophilic and lipophilic moieties of a molecule. See e.g., A. Rabaron et al., Physical methods for measurement of the HLB of ether and ester non-ionic surface active agents: H-NMR and dielectric constant, 99 INT. J. PHARM. 29-36 (1993). The HLB scale ranges from 0 to 20, where higher HLB values correspond to more water-soluble molecules and lower HLB values correspond to more lipid-soluble molecules.

Oils have an HLB value of about 1. Therefore, it should be appreciated that in certain embodiments, oils included in the disclosed pharmaceutical compositions have an HLB value of about 1.

In certain embodiments, the surfactant is an oil-soluble surfactant having an HLB value of from about 2 to about 4. In certain embodiments, the surfactant is a water-dispersible surfactant having an HLB value between about 9 and about 12. In certain embodiments, the surfactant is a water-soluble surfactant having an HLB value of about 12 to about 20. The HLB value of the lipid-based excipient determines what type of lipid-based formulation will be formed, namely oily solubilizers, emulsions, microemulsions, or micelles, as summarized in Table 1 below. Accordingly, in various embodiments, the pharmaceutical compositions of the invention may characterized as an oily solubilizer, emulsion, microemulsion or micelles based composition. Suitable surfactants for use in the disclosed pharmaceutical compositions can be selected on the basis of HLB value in order to prepare the desired lipid-based drug delivery system.

TABLE 1
HLB Value and Formulation Classifications

| Functionality | HLB Value | Lipid-Based Formulation |
|---|---|---|
| Oily Phase | 1 | Oily Solubilizers |
| Water Insoluble | 2-4 | Microemulsions/Emulsion |
| Surfactant | | |
| Water Insoluble | 5-6 | Microemulsions/Emulsions |
| Surfactant | | |
| Wetting Agent | 7-9 | Emulsions |
| Water Dispersible | 10-12 | Microemulsions |
| Surfactant | | |
| Water Soluble Surfactant | 12 | Micelles |

Lipid formulations can be further classified into four main types based on the composition and type of dispersion formed. In one embodiment, the pharmaceutical composition is a Type I formulation, which includes oil excipients that do not form dispersions and require digestion in order to form emulsions and be absorbed.

Alternatively, the pharmaceutical composition is a Type II, IIIA, or IIIB formulation, each of which are mixtures of oils and surfactants that form emulsions with aqueous liquids. For example, the pharmaceutical composition may be a Type II formulation, which include oils and water-insoluble surfactants that form emulsions, including self-emulsifying drug delivery systems (SEDDS). Self-emulsifying drug delivery systems form emulsions on contact with aqueous liquids without mechanical agitation or heating. Exemplary SEDDS for use in the invention described herein include self-emulsifying microemulsion (SMEDDS) or self-emulsifying nanoemulsion (SNEDDS) drug delivery systems, which are distinguishable based on droplet size. Alternatively, the pharmaceutical composition may be a Type IIIA or IIIB formulation, which include oils, water-insoluble and/or water-soluble surfactants and optionally, cosolvents. Exemplary cosolvents include triacetin, ethanol, glycerol, propylene glycol and polyethylene glycols, e.g., (PEG)-400.

Finally, the pharmaceutical composition may be a Type IV formulation, which includes water-soluble surfactants and optionally non-oil cosolvents that form micellar dispersions. The types of lipid-based formulations and their compositions are summarized in Table 2, and exemplary excipients, their HLB values and applications are summarized in Table 3, below.

TABLE 2
Types of Lipid-Based Drug Delivery Systems

| | Composition | Lipid-Based Formulation |
|---|---|---|
| Type I | Oils without surfactants | Non-dispersing |
| Type II | Oils and water insoluble | Emulsion (SEDDS) |
| | surfactants | |
| Type IIIA | Oils, surfactants, cosolvents | Fine emulsion |
| | | (SEDDS and SMEDDS) |
| Type IIIB | Oils, surfactants, cosolvents | Microemulsion |
| | | (SEDDS and SMEDDS) |
| Type IV | Water-soluble surfactants | Micellar dispersion |

TABLE 3
Exemplary Excipients and Their HLB Values and Applications.

| Excipient | HLB Value | Application |
|---|---|---|
| Maisine ® CC | 1 | Oily Vehicle |
| Peceol ™ | 1 | |
| Labrafac ™ PG | 1 | |
| Labrafac ™ Lipophile WL 1349 | 1 | |
| Lauroglycol ™ 90 | 3 | Water Insoluble |
| Plurol ® Oleique CC 497 | 3 | Surfactant |
| Capryol ™ 90 | 5 | (SEDDS & SMEDDS) |
| Geloil ™ SC | 5 | |
| Labrafil ® M 1944 CS | 9 | Water Dispersible |
| Labrafil ® M 2125 CS | 9 | Surfactant |
| Labrasol ® ALF | 12 | (Micellar/Microemulsions) |

### 1.2.1. Oils

Suitable oils for use in the pharmaceutical compositions of the invention described herein include lipids and fatty acids that are derived from vegetable sources via esterification of fatty acids with alcohols, e.g., glycerol, polyglycerol, propylene glycol, and polyethylene glycol, and by the alcoholysis of vegetable oils and fats with glycerol, polyethylene glycol, and propylene glycol.

In some embodiments, oils suitable for inclusion in pharmaceutical compositions of the invention include, but are not limited to, glyceryl monooleate, glyceryl monolinoleate, propylene glycol dicaprolate/dicaprate, soybean oil, polyglyceryl-3 dioleate, oleic acid, glyceryl caprylate, medium chain triglycerides, and combinations thereof.

In a particular embodiment, the pharmaceutical composition includes glyceryl monooleate, e.g., Peceol™ available from Gattefossé, which includes mono-, di-, and triglycerides of oleic (C_{18:1}) acid, the monoester fraction being predominant. Glyceryl monooleate is used as a solubilizer for lipophilic active pharmaceutical ingredients (APIs). Glyceryl monooleate is also used in SEDDS and SMEDDS, as described herein.

Alternatively or in combination, the pharmaceutical compositions of the invention can include glyceryl monolinoleate. Glyceryl monolinoleate, e.g., Maisine® CC available from Gattefossé, is a winterized oil composed of long-chain mono, di- and triglycerides, primarily linoleic (C_{18:2}) and oleic acid (C_{18:1}). Glyceryl monolinoleate is used in lipid-based formulations to solubilize poorly water-soluble lipophilic APIs and is also used in self-emulsifying lipid formulations (SEDDS and SMEDDS). In some embodiments, the pharmaceutical compositions include glyceryl monolinoleate in an amount from about 3% to about 30%, from about 5% to about 25%, or from about 10% to about 20% by weight of the composition. In some embodiments, the pharmaceutical compositions include glyceryl monolinoleate in an amount of at least about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% by weight of the composition. In some embodiments, the pharmaceutical compositions include glyceryl monolinoleate in an amount of at most about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% by weight of the composition. In some embodiments, the pharmaceutical compositions include glyceryl monolinoleate in an amount of about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% by weight of the composition.

Alternatively, or in combination, the pharmaceutical compositions of the invention include propylene glycol. Propylene glycol dicaprolate/dicaprate, e.g., Labrafac™ PG available from Gattefossé, includes propylene glycol esters of caprylic (C₈) and capric (C₁₀) acids. Propylene glycol dicaprolate/dicaprate is also used in lipid-based formulations, SEDDS, and SMEDDS.

The pharmaceutical compositions of the invention described herein may further include medium chain triglycerides. Medium chain triglycerides, e.g., MIGLYOL® 812 available from IOI Oleo GmbH and Labrafac™ Lipophile WL 1349 available from Gattefossé, consists of medium-chain triglycerides of caprylic (C₈) and capric (C₁₀) acids. Medium chain triglycerides are also used in lipid-based formulations, SEDDS and SMEDDS. In some embodiments, the pharmaceutical compositions include medium chain triglycerides in an amount from about 3% to about 30%, from about 5% to about 20%, or from about 10% to about 15% by weight of the composition. In some embodiments, the pharmaceutical compositions include medium chain triglycerides in an amount of at least about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% by weight of the composition. In some embodiments, the pharmaceutical compositions include medium chain triglycerides in an amount of at most about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% by weight of the composition. In some embodiments, the pharmaceutical compositions include medium chain triglycerides in an amount of about 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% by weight of the composition.

In further embodiments, the oil includes caprylic/capric triglyceride. Caprylic/capric triglyceride, e.g., MIGLYOL® 810 and MIGLYOL® 812 available from CREMER OLEO GmbH & Co. KG, are esters of caprylic and capric fatty acids and glycerin derived from saturated coconut and palm kernel oil.

In some embodiments, the oil includes a soybean oil-based excipient. Soybean oil-based excipients, e.g., Geloil™ SC available from Gattefossé, include a mixture of soybean oil, glyceryl distearate (C₁₈) and polyglyceryl-3 dioleate (C_{18:1}). Geloil™ SC serves as a vehicle to suspend pharmaceutical ingredients in soft gelatin capsule and has good dispersibility in aqueous fluid.

In some embodiments the oil includes polyglyceryl-3 dioleate. Polyglyceryl-3 dioleate, e.g., Plurol® Oleique CC 497, includes polyglyceryl-3-esters of oleic acid (C_{18:1}), the diester fraction being predominant. Polyglyceryl-3 dioleate also serves as a co-surfactant in SEDDS and SMEDDS formulations.

In some embodiments, the oil includes oleic acid. Oleic acid is a monounsaturated omega-9 fatty acid (C_{18:1}). In some embodiments, the pharmaceutical compositions include oleic acid in an amount from about 10% to about 40%, from about 15% to about 35%, or from about 20% to about 30% by weight of the composition. In some embodiments, the pharmaceutical compositions include oleic acid in an amount of at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% by weight of the composition. In some embodiments, the pharmaceutical compositions include oleic acid in an amount of at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% by weight of the composition. In some embodiments, the pharmaceutical compositions include oleic acid in an amount of about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% by weight of the composition.

In some embodiments, the oil includes glyceryl caprylate mono- and di-glycerides. Glyceryl caprylate mono- and diglycerides, e.g., IMWITOR® 988 and/or IMWITOR® 742 available from CREMER, includes a blend of glycerol esters of caprylic (C₈H₁₆O₂) acid derived from vegetable sources. In some embodiments, the pharmaceutical compositions include glyceryl caprylate mono- and diglycerides from about 10% to about 40%, from about 20% to about 35%, or from about 20% to about 30%, or about 30% to about 35% by weight of the composition. In alternative embodiments, the pharmaceutical compositions include glyceryl caprylate mono- and diglycerides in an amount from about 15% to about 45%, from about 25% to about 40%, or from about 30% to about 35% by weight of the composition. In some embodiments, the pharmaceutical compositions include glyceryl caprylate mono- and diglycerides in an amount of at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, or 45% by weight of the composition. In some embodiments, the pharmaceutical compositions include glyceryl caprylate mono- and diglycerides in an amount of at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, or 45% by weight of the composition. In some embodiments, the pharmaceutical compositions include glyceryl caprylate mono- and diglycerides in an amount of about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, or 45% by weight of the composition.

In some embodiments, the total oil content of the pharmaceutical composition is about 10% to about 80%, about 15% to about 70%, about 20% to about 60%, or about 30% to about 50% by weight of the composition. In some embodiments, the total oil content of the pharmaceutical composition is at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition. In some embodiments, the total oil content of the pharmaceutical composition is at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition. In some embodiments, the total oil content of the pharmaceutical composition is about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition.

In some embodiments, pharmaceutical compositions include one oil. In some embodiments, pharmaceutical compositions include two, three, four, five, or more oils. In an exemplary embodiment, the pharmaceutical composition includes two oils, e.g., medium chain triglycerides and glyceryl caprylate mono- and diglycerides. In another exemplary embodiment, the pharmaceutical composition includes three oils, e.g., medium chain triglycerides, glyceryl caprylate mono- and diglycerides, and oleic acid. In some embodiments, the pharmaceutical compositions do not include an oil as described herein.

### 1.2.2. Surfactants

Surfactants can be added to the pharmaceutical compositions disclosed herein, for example, to prepare self-emulsifying, self-microemulsifying drug delivery systems, and self-nanoemulsifying drug delivery systems.

Suitable surfactants for use in the pharmaceutical compositions of the invention described herein include oleoyl polyoxyl-6 glycerides, linoleoyl polyoxyl-6 glycerides, caprylocaproyl polyoxyl-8 glycerides, polysorbate 80, polyoxyl 40 hydrogenated castor oil, polyoxyl 15 hydroxystearate, lauroyl polyoxyl-32 glycerides, and combinations thereof.

In certain embodiments, the pharmaceutical compositions of the invention include oleoyl polyoxyl-6 glycerides. Oleoyl polyoxyl-6 glycerides, e.g., Labrafil® M 1944 CS available from Gattefossé, comprise mono-, di-, and triglycerides and PEG-6 (MW 300) mono- and diesters of oleic (C_{18:1}) acid. Oleoyl polyoxyl-6 glycerides are used to solubilize poorly-soluble APIs. Oleoyl polyoxyl-6 glycerides are also used in single excipient formulation systems to prepare SEDDS and can form SMEDDS when combined with high HLB surfactants, e.g., Labrasol® ALF or Gelucire® 44/14.

In some embodiments, the surfactant includes linoleoyl polyoxyl-6 glycerides. Linoleoyl polyoxyl-6 glycerides, e.g., Labrafil® M 2125 CS available from Gattefossé, comprise mono-, di-, and triglycerides and PEG-6 (MW 300) mono- and diesters for linoleic (C_{18:2}) acid. Linoleoyl polyoxyl-6 glycerides solubilize poorly water-soluble APIs in lipid-based formulations. Linoleoyl polyoxyl-6 glycerides also self-emulsify in aqueous media forming a coarse dispersion, i.e., SEDDS, and forms SMEDDS in pharmaceutical compositions that also include surfactants such as Labrasol® ALF or Gelucire® 44/14.

In some embodiments, the surfactant includes caprylocaproyl polyoxyl-8 glycerides. Caprylocaproyl polyoxyl-8 glycerides, e.g., Labrasol® ALF available from Gattefossé, comprise a small fraction of mono-, di- and triglycerides and mainly PEG-8 (MW 400) mono- and diesters of caprylic (C₈) and capric (C₁₀) acids. Caprylocaproyl polyoxyl-8 glycerides are a solubilizer for poorly-soluble APIs. Caprylocaproyl polyoxyl-8 glycerides are also used in single excipient formulation systems that self-emulsify in aqueous fluid into microemulsions (SMEDDS). In some embodiments, the pharmaceutical composition includes caprylocaproyl polyoxyl-8 glycerides in an amount from about 50% to about 80%, from about 55% to about 70%, or from about 60% to about 65% by weight of the composition. In some embodiments, the pharmaceutical composition includes caprylocaproyl polyoxyl-8 glycerides in an amount of at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition. In some embodiments, the pharmaceutical composition includes caprylocaproyl polyoxyl-8 glycerides in an amount of at most about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition. In some embodiments, the pharmaceutical composition includes caprylocaproyl polyoxyl-8 glycerides in an amount of about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition.

In some embodiments, the surfactant includes propylene glycol monolaurate. Propylene glycol monolaurate, e.g., Lauroglycol™ 90, includes propylene mono- and diesters of auric (Cu) acid, mainly monoesters with a small fraction of diesters. Propylene glycol monolaurate is used as a cosurfactant in SEDDS and SMEDDS.

In some embodiments, the surfactant includes propylene glycol monocaprylate. Propylene glycol monocaprylate, e.g., Capryol™ 90 available from Gattefossé, includes propylene glycol esters of acrylic acid (C₈), primarily monoesters and a small fraction of diesters. Propylene glycol monocaprylate is a nonionic water-insoluble surfactant that is used as a cosurfactant in SEDDS and SMEDDS.

It should be appreciated that some embodiments of the pharmaceutical composition include one or more cosurfactants. For example, some embodiments of the pharmaceutical composition include up to 20% (w/w) cosurfactant (e.g., Capryol™ (propylene glycol caprylate) and/or Lauroglycol™ (Propylene glycol monolaurate)).

Suitable surfactants also include polysorbate 80 (e.g., TWEEN® 80 from Croda International Plc), polyoxyethylene sorbitan trioleate (e.g., TWEEN® 85 from Croda International Plc), PEG-35 castor oil, polyoxyl 40 hydrogenated castor oil (e.g., KOLLIPHOR® RH 40), polyoxyl 15 hydroxystearate (e.g., KOLLIPHOR® HS 15), lauroyl polyoxyl-32 glycerides (e.g., GELUCIRE® 44/14), and/or Vitamin E TPGS.

In some embodiments, the surfactant includes TWEEN® 80 from about 15% to about 50%, from about 20% to about 40%, from about 30% to about 35% by weight of the composition. In some embodiments, the composition includes TWEEN® 80 in amount of at least about 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% by weight of the composition. In some embodiments, the composition includes TWEEN® 80 in amount of at most about 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% by weight of the composition. In some embodiments, the composition includes TWEEN® 80 in an amount of about 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% by weight of the composition.

In some embodiments, the surfactant includes polyoxyl 40 hydrogenated castor oil, e.g., KOLLIPHOR® RH40. In some embodiments, the polyoxyl 40 hydrogenated castor oil is KOLLIPHOR® RH 40. KOLLIPHOR® RH 40 is a digestible surfactant. In some embodiments, the pharmaceutical compositions include polyoxyl 40 hydrogenated castor oil in an amount from about 10% to about 80%, from about 20% to about 70%, or from about 25% to about 65% by weight of the composition. In alternative embodiments, the pharmaceutical compositions include polyoxyl 40 hydrogenated castor oil in an amount from about 15% to about 45%, from about 25% to about 40%, or from about 30% to about 40% by weight of the composition. In some embodiments, the pharmaceutical compositions include polyoxyl 40 hydrogenated castor oil in an amount of at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition. In some embodiments, the pharmaceutical compositions include polyoxyl 40 hydrogenated castor oil in an amount of at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition. In some embodiments, the pharmaceutical compositions include polyoxyl 40 hydrogenated castor oil in an amount of about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition.

In some embodiments, the surfactant includes polyoxyl 15 hydroxystearate from about 15% to about 50%, from about 20% to about 40%, from about 30% to about 35% by weight of the composition. In some embodiments, polyoxyl 15 hydroxy stearate is KOLLIPHOR® HS 15. KOLLIPHOR® HS 15 is a non-digestible surfactant. In some embodiments, the composition includes polyoxyl 15 hydroxystearate (e.g., KOLLIPHOR® HS 15) in amount of at least about 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% by weight of the composition. In some embodiments, the composition includes polyoxyl 15 hydroxystearate (e.g., KOLLIPHOR® HS 15) in amount of at most about 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% by weight of the composition. In some embodiments, the composition includes polyoxyl 15 hydroxystearate (e.g., KOLLIPHOR® HS 15) in amount of about 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% by weight of the composition.

In some embodiments, the surfactant includes lauroyl polyoxyl-32 glycerides, e.g., GELUCIRE® 44/14. In some embodiments, the lauroyl polyoxyl-32 glycerides is GELUCIRE® 44/14. In some embodiments, the pharmaceutical compositions include lauroyl polyoxyl-32 glycerides in an amount from about 10% to about 80%, from about 20% to about 70%, or from about 25% to about 65% by weight of the composition. In alternative embodiments, the pharmaceutical compositions include lauroyl polyoxyl-32 glycerides in an amount from about 15% to about 45%, from about 25% to about 40%, or from about 30% to about 40% by weight of the composition. In some embodiments, the pharmaceutical compositions include lauroyl polyoxyl-32 glycerides in an amount of at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition. In some embodiments, the pharmaceutical compositions include lauroyl polyoxyl-32 glycerides in an amount of at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition. In some embodiments, the pharmaceutical compositions include lauroyl polyoxyl-32 glycerides in an amount of about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition.

In some embodiments, the pharmaceutical composition includes Vitamin E TPGS, which may behave as both a surfactant and a stabilizer. When used as a surfactant, Vitamin E TPGS may be provided in amount from about 10% to about 80% or from about 20% to about 70% by weight of the composition. In some embodiments, the pharmaceutical compositions include Vitamin E TPGS in an amount of at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition. In some embodiments, the pharmaceutical compositions include Vitamin E TPGS in an amount of at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition. In some embodiments, the pharmaceutical compositions include Vitamin E TPGS in an amount of about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight of the composition.

### 1.3. Additives

In some embodiments, the compositions described herein may include an additive. In some embodiments, the additive may be a stabilizer such as butylated hydroxytoluene (BHT), butylated hydroxyanisole (BHA), propyl gallate, ascorbic acid-6-palmitate, alpha tocopherol, Vitamin E TPGS (when provided as a stabilizer rather than a surfactant), or a combination thereof. In some embodiments, the stabilizer (e.g., BHT) may be provided in an amount of about 0.01% to about 10% by weight of the composition. In some embodiments, the stabilizer (e.g., BHT) may be provided in an amount of at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10% by weight of the composition. In some embodiments, the stabilizer (e.g., BHT) may be provided in an amount of at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10% by weight of the composition. In some embodiments, the stabilizer (e.g., BHT) may be provided in an amount of about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10% by weight of the composition.

For example, butylated hydroxyanisole (BHA) may be provided in an amount of up to about 0.05% by weight of the composition. Propyl gallate may be provided in an amount of up to about 0.1% by weight of the composition. Ascorbic acid-6-palmitate may be provided in an amount of up to about 3% by weight of the composition. Alpha tocopherol may be provided in an amount of up to about 4% by weight of the composition.

### 1.4. Cosolvents

As described herein, in some embodiments, the pharmaceutical compositions may include a cosolvent. In some embodiments, the cosolvent may be triacetin, ethanol, glycerol, propylene glycol, polyethylene glycol (e.g., PEG-400), or a combination thereof. In some embodiments, the cosolvent includes ethanol. In some embodiments, the cosolvent (e.g., ethanol) may be provided in an amount of about 1% to about 20%, or about 1% to about 10%, or about 5% to about 15% by weight of the composition. In some embodiments, the cosolvent (e.g., ethanol) may be provided in an amount of at least about 1, 2, 3, 4, 5, 6, 7, 8, 9, 10, 11, 12, 13, 14, 15, 16, 17, 18, 19, or 20% by weight of the composition. In some embodiments, the cosolvent (e.g., ethanol) may be provided in an amount of at most about 1, 2, 3, 4, 5, 6, 7, 8, 9, 10, 11, 12, 13, 14, 15, 16, 17, 18, 19, or 20% by weight of the composition. In some embodiments, cosolvent (e.g., ethanol) may be provided in an amount of about 1, 2, 3, 4, 5, 6, 7, 8, 9, 10, 11, 12, 13, 14, 15, 16, 17, 18, 19, or 20% by weight of the composition.

### 1.5. Enteric Delivery

In some embodiments, the pharmaceutical compositions disclosed herein are formulated for enteric delivery. Enteric drug delivery vehicles, e.g., coatings, capsules, and other encapsulation technologies, are used to protect acid sensitive APIs from the stomach's low pH environment, to protect the stomach from irritating APIs, and to facilitate colonic drug delivery.

The delayed release of drugs from enteric formulations arises from the insolubility of enteric polymers at low pH values. Enteric polymers dissolve at a pH values of about 5.0-5.5 and higher. Enteric formulations can also be affected by factors, such as the nature of the API (e.g., whether the API is ionic), the thickness of the coating/capsule shell, the presence of imperfections (e.g., cracks, holes, etc.), the properties of the polymer(s) used (e.g., dissolution rate at relevant pHs), and agitation rate.

Enteric polymers, for use in the invention described herein, include polyacids, such as cellulose acetate phthalate, cellulose acetate trimellitate, polyvinyl acetate phthalate, hydroxypropylmethylcellulose phthalate, hydroxypropylmethyl cellulose acetate succinate, methacrylate-ethylacrylate copolymers, and methacrylate-methylmethacrylate copolymers.

To target the colon, a combination of pH-triggered (e.g., at pH 6.8-7.2) and enzyme-triggered polymers can be used. Additionally, capsules-in-capsules and coated or uncoated capsules including liquid filled hard capsules can be used to target colonic delivery.

Suitable enteric capsules for use in the pharmaceutical compositions of the disclosure include gelatin and EUDRAGIT® L 100-based capsules as described in U.S. Pat. No. 8,685,445 and hydroxypropyl methylcellulose acetate succinate-based capsules as described in US 20130295188A1. Enteric coated capsules are also contemplated. See, e.g., U.S. Pat. Nos. 4,518,433, 4,816,259, and 5,330,759. In some embodiments, enteric capsules are Vcaps® Enteric Capsules from Capsugel.

Enteric coated methylnaltrexone formulations have yielded unpredictable results. For example, while an enteric coated methylnaltrexone formulation more effectively reduced oral-cecal delay caused by morphine than an uncoated formulation (laxation data was not reported) (see, e.g., U.S. Pat. No. 6,274,591), capsules containing enterically coated spheroids of a formulation of methylnaltrexone surprisingly did not induce laxation in patients suffering from opioid-induced constipation (see, e.g., U.S. Pat. No. 8,524,276).

### 2. Administration

Pharmaceutical compositions may be administered to a patient as required to provide an effective amount of an ion pair of methylnaltrexone with docusate or lauryl sulfate, as described herein.

In certain embodiments, the patient is orally administered the pharmaceutical composition as described herein at least once a day. In certain embodiments, the patient is orally administered the pharmaceutical composition as described herein at least twice a day. In certain embodiments, the patient is orally administered the pharmaceutical composition as described herein at least three times a day. In other embodiments, the patient is orally administered the pharmaceutical composition up to once a day. In other embodiments, the patient is orally administered the pharmaceutical composition up to twice a day. In other embodiments, the patient is orally administered the pharmaceutical composition up to three times a day. In certain embodiments, the patient is orally administered the pharmaceutical composition not more than once a day. In certain embodiments, the patient is orally administered the pharmaceutical composition not more than twice a day. In certain embodiments, the patient is orally administered the pharmaceutical composition not more than three times a day. In certain embodiments, the patient is orally administered the pharmaceutical composition as needed. In certain embodiments, the patient is orally administered the pharmaceutical composition as needed, but not more than once a day. In certain embodiments, the patient is orally administered the pharmaceutical composition as needed, but not more than twice a day. In certain embodiments, the patient is orally administered the pharmaceutical composition as needed, but not more than three times a day.

For example, a liquid dosage form of a provided pharmaceutical composition may be orally administered to a patient in a single day, for example, a unit dosage of about 1 mg to about 100 mg, about 50 mg to about 900 mg, about 75 mg to about 850 mg, about 100 mg to about 850 mg, about 150 mg to about 850 mg, about 200 mg to about 800 mg, or about 200 mg to about 700 mg of the ion pair. In other embodiments, the pharmaceutical compositions may be orally administered to a patient in a single day, for example, at a unit dosage of at least about 1 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, or about 900 mg of the ion pair. In other embodiments, the pharmaceutical compositions may be orally administered to a patient in a single day, for example, at a unit dosage of at most about 1 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, or about 900 mg of the ion pair. In other embodiments, the pharmaceutical compositions may be orally administered to a patient in a single day, for example, at a unit dosage of about 1 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, or about 900 mg of the ion pair.

In some embodiments, the invention described herein provides a method for treating an opioid-induced side effect in a patient in need thereof, comprising the step of orally administering to said patient one or more capsules wherein said liquid oral dosage forms provide about 1 mg to about 100 mg, about 50 mg to about 900 mg, about 75 mg to about 850 mg, about 100 mg to about 850 mg, about 150 mg to about 850 mg, about 200 mg to about 800 mg, or about 200 mg to about 700 mg of the ion pair. In some embodiments, the invention described herein provides a method for treating an opioid-induced side effect in a patient in need thereof, comprising the step of orally administering to said patient one or more capsules wherein said liquid oral dosage forms provide at least about 1 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, or about 900 mg of the ion pair. In some embodiments, the invention described herein provides a method for treating an opioid-induced side effect in a patient in need thereof, comprising the step of orally administering to said patient one or more capsules wherein said liquid oral dosage forms provide at most about 1 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, or about 900 mg of the ion pair. In some embodiments, the invention described herein provides a method for treating an opioid-induced side effect in a patient in need thereof, comprising the step of orally administering to said patient one or more capsules wherein said liquid oral dosage forms provide about 1 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, or about 900 mg of the ion pair.

In certain embodiments, a single capsule formulation of the invention described herein provides about 1 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, or about 900 mg of methylnaltrexone-lauryl sulfate or methylnaltrexone-docusate ion pairs. In certain embodiments, a single capsule formulation of the invention described herein provides at least about 1 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, or about 900 mg of methylnaltrexone-lauryl sulfate or methylnaltrexone-docusate ion pairs. In certain embodiments, a single capsule formulation of the invention described herein provides at most about 1 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, or about 900 mg of methylnaltrexone-lauryl sulfate or methylnaltrexone-docusate ion pairs.

As defined above, in certain embodiments the term “effective amount,” as used in connection with an amount of methylnaltrexone ion pairs, means an amount of methylnaltrexone ion pair sufficient to achieve the desired treatment, for example, to achieve laxation in a patient. In some embodiments, an effective amount means an amount of methylnaltrexone ion pair sufficient to achieve laxation in a patient within about 24 hours, within about 12 hours, within about 8 hours, within about 5 hours, within about 4 hours, within about 3 hours, within about 2 hours, or within about 1 hours of administration to said patient. In some embodiments, effective amount means an amount of methylnaltrexone ion pair sufficient to achieve laxation within about 4 hours of administration to the patient. In some embodiments, effective amount means an amount of methylnaltrexone ion pair sufficient to achieve laxation within about 4 hours of administration to the patient for at least 99%, at least 95%, at least 90%, at least 85%, at least 80%, at least 75%, or at least 50% of all doses administered. In some embodiments, effective amount means an amount of methylnaltrexone ion pair sufficient to achieve laxation within about 4 hours of administration to the patient for all doses administered during first four weeks of dosing.

In some embodiments, the pharmaceutical compositions are administered to a fasted patient. As used herein, the term “fasted” means that the patient has not eaten any food for at least 2 hours, for at least 4 hours, for at least 6 hours, for at least 8 hours, for at least 10 hours, or for at least 12 hours prior to administration of a provided formulation. In certain embodiments, the term “fasted” means an overnight fast. It is believed that improved effects will be seen in fasted patients than in fed patients. These effects may be magnified in patients administered liquid methylnaltrexone ion pair pharmaceutical compositions provided in an encapsulated form, e.g., soft gel capsules, hard gel capsules, and enteric gel capsules.

In other embodiments, the pharmaceutical compositions are administered to a patient that has not fasted. Therefore, there is no requirement that the patient not have eaten before pharmaceutical compositions are administered.

### 3. Combination Products and Combined Administration

It will also be appreciated that the pharmaceutical compositions provided herein can be employed in combination therapies, that is, provided pharmaceutical compositions can be administered concurrently with, prior to, or subsequent to, one or more other desired therapeutics or medical procedures. Particular combination therapies (therapeutics or procedures) to employ in a combination regimen will take into account compatibility of the desired therapeutics and/or procedures and the desired therapeutic effect to be achieved. It will also be appreciated that therapies employed may achieve a desired effect for the same disorder (for example, a formulation may be administered concurrently with another compound used to treat the same disorder), or they may achieve different effects (e.g., control of any adverse effects). As used herein, additional therapeutic compounds which are normally administered to treat or prevent a particular disease, or condition, are known as “appropriate for the disease, or condition, being treated.”

In certain embodiments, pharmaceutical compositions of the disclosure and one or more other active agents may be administered together in a single formulation (e.g., unit dosage form); in other embodiments, pharmaceutical compositions and one or more other active agents may be administered as separate pharmaceutical compositions. In certain embodiments, methylnaltrexone ion pairs and/or one or more other active agents may be administered in multiple doses.

In other embodiments, the other active agent administered in combination with a methylnaltrexone ion pair or formulation of the invention is an opioid. Combination therapy of methylnaltrexone ion pairs and an opioid can allow simultaneous relief of pain and minimization of opioid-associated side effects (e.g., gastrointestinal effects, such as delayed gastric emptying, and altered GI tract motility). Accordingly, in certain embodiments, the invention described herein provides a unit dosage form comprising a combination of methylnaltrexone ion pairs with an opioid together in a liquid oral dosage form (e.g., a capsule) suitable for oral administration.

Opioids useful for analgesia are known in the art. For example, opioid compounds include, but are not limited to, alfentanil, anileridine, asimadoline, bremazocine, burprenorphine, butorphanol, codeine, dezocine, diacetylmorphine (heroin), dihydrocodeine, diphenoxylate, ethylmorphine, fedotozine, fentanyl, funaltrexamine, hydrocodone, hydromorphone, levallorphan, levomethadyl acetate, levorphanol, loperamide, meperidine (pethidine), methadone, morphine, morphine-6-glucoronide, nalbuphine, nalorphine, nicomorphine, opium, oxycodone, oxymorphone, papaveretum, pentazocine, propiram, propoxyphene, remifentanyl, sufentanil, tilidine, trimebutine, and tramadol. In some embodiments the opioid is at least one opioid selected from alfentanil, buprenorphine, butorphanol, codeine, dezocine, dihydrocodeine, fentanyl, hydrocodone, hydromorphone, levorphanol, meperidine (pethidine), methadone, morphine, nalbuphine, nicomorphine, oxycodone, oxymorphone, papaveretum, pentazocine, propiram, propoxyphene, sufentanil and/or tramadol. In certain embodiments of the invention described herein, the opioid is selected from morphine, codeine, oxycodone, hydrocodone, dihydrocodeine, propoxyphene, fentanyl, tramadol, and mixtures thereof. In a particular embodiment, the opioid is loperamide. In other embodiments, the opioid is a mixed agonist such as butorphanol. In some embodiments, the subjects are administered more than one opioid, for example, morphine and heroin or methadone and heroin.

Typically, the amount of other active agent(s) administered in combination therapy may be no more than the amount that would normally be administered in monotherapy with the relevant agent(s). In certain embodiments, the amount of other active agent administered in combination therapy may be less than that normally administered in monotherapy with the relevant agent(s). For example, in certain embodiments of the invention described herein, the amount of additional active agent can range from about 50% to about 100% of the amount normally present in a formulation comprising that compound as the only therapeutic agent.

In certain embodiments, pharmaceutical compositions may also be used in conjunction with and/or in combination with conventional therapies for gastrointestinal dysfunction to aid in the amelioration of constipation and bowel dysfunction. For example, conventional therapies include, but may not be limited to functional stimulation of the intestinal tract, stool softening agents, laxatives (e.g., diphelymethane laxatives, cathartic laxatives, osmotic laxatives, saline laxatives), bulk forming agents and laxatives, lubricants, intravenous hydration, and nasogastric decompression.

### 4. Uses and Kits of Pharmaceutical Compositions

The invention described herein provides pharmaceutically acceptable compositions as described herein comprising methylnaltrexone with docusate or lauryl sulfate, for oral administration useful for the delivery of such pharmaceutical compositions in any context in which such delivery is desirable. In certain embodiments, provided pharmaceutical compositions are useful for the delivery of methylnaltrexone, e.g., as an ion pair with docusate or lauryl sulfate, in antagonizing undesirable side effects of opioid analgesic therapy (e.g., gastrointestinal effects (e.g., delayed gastric emptying, altered GI tract motility)). Furthermore, pharmaceutical compositions may be used to treat subjects having disease states that are ameliorated by binding μ opioid receptors, or in any treatment wherein temporary suppression of the μ opioid receptor system is desired (e.g., ileus). In certain embodiments of the invention described herein, the methods are for use in human subjects.

Accordingly, administration of provided pharmaceutical compositions may be advantageous for treatment, prevention, amelioration, delay or reduction of side effects of opioid use, such as, for example, gastrointestinal dysfunction (e.g., inhibition of intestinal motility, constipation, GI sphincter constriction, nausea, emesis (vomiting)), biliary spasm, opioid bowel dysfunction, colic, dysphoria, pruritus, urinary retention, depression of respiration, papillary constriction, cardiovascular effects, chest wall rigidity and cough suppression, depression of stress response, and immune suppression associated with use of narcotic analgesia, or combinations thereof. Use of a pharmaceutical composition may thus be beneficial from a quality of life standpoint for subjects undergoing use of opioids, as well as to reduce complications arising from chronic constipation, such as hemorrhoids, appetite suppression, mucosal breakdown, sepsis, colon cancer risk, and myocardial infarction.

In some embodiments, provided pharmaceutical compositions are useful for administration to a subject undergoing acute opioid use. In some embodiments, provided pharmaceutical compositions are useful for administration to patients suffering from post-operative gastrointestinal dysfunction.

In certain embodiments, provided pharmaceutical compositions are also useful for administration to subjects undergoing chronic opioid use (e.g., terminally ill patients receiving opioid therapy such as an AIDS patient, a cancer patient, a cardiovascular patient; subjects receiving chronic opioid therapy for pain management; subjects undergoing opioid therapy for maintenance of opioid withdrawal). In some embodiments, the subject is a subject using opioid therapy for chronic pain management. In certain embodiments, the pain is non-malignant pain (e.g., back pain, neuropathic pain, pain associated with fibromyalgia, osteoarthritis). In some embodiments, the subject is a terminally ill patient. In other embodiments, the subject is a person undergoing opioid withdrawal maintenance therapy.

In certain embodiments, the pharmaceutical compositions provided herein are administered to subjects that have been selected for treatment. In specific embodiments, the subject is selected based on the subject having an increased risk for developing one or more of the conditions set forth above. In another embodiment, the subject is selected based on the use of opioid therapy for pain management, or based on having one or more of the conditions set forth herein. In certain embodiments, the subject is constipated or has a history of constipation due to opioid therapy. In one embodiment, a constipated subject has not had a bowel movement in the previous three days. In one embodiment, a constipated subject has had less than three bowel movements in the previous week. In certain embodiments, a constipated subject has had less than three rescue-free bowel movements per week on average over the last four consecutive weeks, and one or more of the following: (a) hard or lumpy stools, (b) straining during bowel movements, and/or (c) sensation of incomplete evacuation after bowel movements.

In certain embodiments, the subject is selected for treatment with a pharmaceutical composition described herein based on the use of opioids, e.g., for non-malignant pain. The subject may be using opioids intermittently or regularly. In one embodiment, the subject that is selected has been taking opioids as needed. In one embodiment, the subject that is selected has been taking opioids for less than one week. In one embodiment, the subject that is selected has been taking opioids over the course of at least one week. In another embodiment, the subject that is selected has been taking opioids over the course of at least two weeks. In another embodiment, the subject that is selected has been taking opioids over the course of at least three weeks. In another embodiment, the subject that is selected has been taking opioids over the course of at least four weeks. In another embodiment, the subject that is selected has been taking opioids over the course of at least three months. In another embodiment, the subject that is selected has been taking opioids over the course of at least six months. In another embodiment, the subject that is selected has been taking opioids over the course of at least twelve months. In another embodiment, the subject that is selected has been taking opioids over the course of more than one year. In another embodiment, the subject that is selected has been taking opioids at least every other day over the course of at least two weeks. In one embodiment, the subject that is selected has been receiving at least 7 doses of at least 25 mg of oral morphine equivalents over at least 14 days. In one embodiment, the subject that is selected has been receiving a daily dose of at least 50 mg of oral morphine equivalents for at least 14 days. In one embodiment, the subject that is selected is constipated due to opioid therapy and has been receiving a daily dose of at least 50 mg of oral morphine equivalents for at least 14 days. In certain embodiments, the subject has been receiving a daily dose of at least 50 mg of oral morphine equivalents for at least 14 days; and has had less than three (3) rescue-free bowel movements per week on average over the least four consecutive weeks that were associated with one or more of the following: (a) a Bristol Stool Form Scale type 1 or 2 for at least 25% of the rescue-free bowel movements, (b) straining during at least 25% of the rescue-free bowel movements; and/or (c) a sensation of incomplete evacuation after at least 25% of the rescue-free bowel movements. A rescue-free bowel movement refers to a bowel movement associated with no laxative use within the 24 hours prior to the bowel movement.

In certain embodiments, the subject selected for treatment with a pharmaceutical composition described herein is a subject suffering from opioid-induced constipation. In certain embodiments, the subject selected for treatment with a pharmaceutical composition described herein is a subject with advanced illness who is receiving palliative care and is suffering from opioid-induced constipation. In certain embodiments, the subject selected for treatment with a pharmaceutical composition described herein is a subject with advanced illness who is receiving palliative care and is suffering from opioid-induced constipation where response to laxative therapy (e.g., bisacodyl, senokot, docusate) has not been sufficient. In certain embodiments, the subject selected for treatment with a pharmaceutical composition described herein is a subject with non-malignant pain who is suffering from opioid-induced constipation. In certain embodiments, the subject selected for treatment with a pharmaceutical composition described herein is a subject with non-malignant pain who is suffering from opioid-induced constipation where response to laxative therapy (e.g., bisacodyl, senokot, docusate) has not been sufficient. In certain embodiments, the subject selected for treatment with a pharmaceutical composition described herein has not responded to standard laxative therapy. In certain embodiments, the subject selected for treatment with a pharmaceutical composition described herein has responded to standard laxative therapy. In certain embodiments, the subject selected for treatment with a pharmaceutical composition described herein is concurrently administered laxative therapy.

Alternative or additional uses for provided pharmaceutical compositions described herein are useful for treating effects of opioid use including, e.g., aberrant migration or proliferation of endothelial cells (e.g., vascular endothelial cells), increased angiogenesis, and increase in lethal factor production from opportunistic infectious agents (e.g., Pseudomonas aeruginosa). Additional advantageous uses of the pharmaceutical compositions descried herein include treatment of opioid-induced immune suppression, inhibition of angiogenesis, inhibition of vascular proliferation, treatment of pain, treatment of inflammatory conditions such as inflammatory bowel syndrome, treatment of infectious diseases and diseases of the musculoskeletal system such as osteoporosis, arthritis, osteitis, periostitis, myopathies, and treatment of autoimmune diseases.

In certain embodiments, provided pharmaceutical compositions may be used in methods for preventing, inhibiting, reducing, delaying, diminishing or treating gastrointestinal dysfunction, including, but not limited to, irritable bowel syndrome, opioid-induced bowel dysfunction, colitis, post-operative or postpartum ileus, nausea and/or vomiting, decreased gastric motility and emptying, inhibition of the stomach, and small and/or large intestinal propulsion, increased amplitude of non-propulsive segmental contractions, constriction of sphincter of Oddi, increased anal sphincter tone, impaired reflex relaxation with rectal distention, diminished gastric, biliary, pancreatic or intestinal secretions, increased absorption of water from bowel contents, gastro-esophageal reflux, gastroparesis, cramping, bloating, abdominal or epigastric pain and discomfort, constipation, idiopathic constipation, post-operative gastrointestinal dysfunction following abdominal surgery (e.g., hysterectomy and colectomy, including for example, right hemicolectomy, left hemicolectomy, transverse hemicolectomy, colectomy takedown, or low anterior resection), and delayed absorption of orally administered medications or nutritive substances.

Provided pharmaceutical compositions are also useful in treatment of conditions including cancers involving angiogenesis, immune suppression, sickle cell anemia, vascular wounds, retinopathy, inflammation associated disorders (e.g., irritable bowel syndrome), immune suppression, and chronic inflammation.

In other embodiments, provided pharmaceutical compositions are useful in preparation of medicaments, including, but not limited to medicaments useful in the treatment of side effects of opioid use, including gastrointestinal side effects (e.g., inhibition of intestinal motility, GI sphincter constriction, constipation), nausea, emesis, vomiting, dysphoria, pruritus, or a combination thereof. Provided pharmaceutical compositions are useful for preparations of medicaments, useful in treatment of patients receiving acute opioid therapy (e.g., patients suffering from post-operative gastrointestinal dysfunction receiving acute opioid administration) or subjects using opioids chronically (e.g., terminally ill patients receiving opioid therapy such as an AIDS patient, a cancer patient, a patient with cardiovascular disease; subjects receiving chronic opioid therapy for pain management (malignant or non-malignant pain); or subjects undergoing opioid therapy for maintenance of opioid withdrawal). Still further, preparation of medicaments useful in the treatment of pain, treatment of inflammatory conditions such as inflammatory bowel syndrome, treatment of infectious diseases, treatment of diseases of the musculoskeletal system such as osteoporosis, arthritis, osteitis, periostitis, myopathies, treatment of autoimmune diseases and immune suppression, therapy of post-operative gastrointestinal dysfunction following abdominal surgery (e.g., colectomy (such as right hemicolectomy, left hemicolectomy, transverse hemicolectomy, colectomy takedown, low anterior resection), idiopathic constipation, and ileus (such as post operative ileus, post partum ileus), and treatment of disorders such as cancers involving angiogenesis, chronic inflammation and/or chronic pain, sickle cell anemia, vascular wounds, and retinopathy.

In still further embodiments, veterinary applications (e.g., treatment of domestic animals, e.g., horse, dogs, cats) of the disclosed pharmaceutical compositions are provided. Thus, use of provided pharmaceutical compositions in veterinary applications analogous to those discussed above for human subjects is contemplated. For example, inhibition of equine gastrointestinal motility, such as colic and constipation, may be fatal to a horse. Resulting pain suffered by the horse with colic can result in a death-inducing shock, while a long-term case of constipation may also cause a horse's death. Treatment of equines with peripheral opioid receptor antagonists has been described, e.g., in US 20050124657.

Still further encompassed by the invention are pharmaceutical packs and/or kits comprising pharmaceutical compositions described herein, and a container (e.g., a foil or plastic package, or other suitable container). Optionally instructions for use are additionally provided in such kits.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

Furthermore, the transitional terms “comprising”, “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed invention. All embodiments described herein that encompass the invention can, in alternate embodiments, be more specifically defined by any of the transitional terms “comprising,” “consisting essentially of,” and “consisting of.”

In order that the invention described herein may be more fully understood, the following examples are set forth. It should be understood that these examples are for illustrative purposes only and are not to be construed as limiting this invention in any manner.

All features of each of the aspects of the invention apply to all other aspects mutatis mutandis. The contents of all references, patents, pending patent applications and published patents, cited throughout this application are hereby expressly incorporated by reference.

### Examples

### 1. Synthesis and Characterization of Ion Pairs.

### 1.1. Methylnaltrexone

Methylnaltrexone may be prepared according to the methods described in detail in U.S. Pat. No. 7,674,904, or obtained from commercial sources such as Covidien, Saint Louis, Mo.

### 1.2. Methylnaltrexone Lauryl Sulfate Ion Pair

The methylnaltrexone lauryl sulfate ion pair was prepared by mixing methylnaltrexone bromide and sodium lauryl sulfate (molar ratio 1:1) in water. The mixing provided a colloidal suspension. Insoluble material was separated from the liquid by centrifugation. The liquid phase was decanted and the wet solids obtained from the aqueous suspension after centrifugation were dissolved in ethanol and the water was removed by azeotropic drying. The dry residue was further dried in a vacuum oven to obtain a solid powder. The product was analyzed by HPLC and found to contain up to 61% (w/w) methylnaltrexone bromide equivalent.

### 1.3. Methylnaltrexone Docusate Ion Pair

An aqueous solution of methylnaltrexone bromide was slowly added to an aqueous solution of sodium docusate with stirring. A milky white suspension was formed indicating the formation of an ion pair. The resulting white, insoluble material was extracted twice with ethyl acetate. The combined ethyl acetate layer was washed once with water and concentrated by rotary evaporation to obtain a foamy solid. Following rotary evaporation, the residue was dried in a vacuum oven at 60° C. to obtain the dry powder. The dry powder was equivalent to 46% (w/w) methylnaltrexone bromide as determined by HPLC.

### 1.4. Lipophilicity

The partition coefficient between octanol and water (Log P) of each ion pair prepared as described herein is summarized in following table:

| | LogP |
|---|---|
| Methylnaltrexone - Lauryl Sulfate | 1.23 |
| Methylnaltrexone - Docusate | 1.67 |

To determine the partition coefficient of each ion pair, about 15 mg of each ion pair was dissolved in separate 100 ml portions of n-octanol (previously saturated with water). Three n-octanol solutions were prepared for each ion pair. Mixtures having three different volumetric ratios of n-octanol to water (9:1, 7:3, and 1:1) were prepared by adding water and agitating the mixtures on a bench top agitator for two hours. After agitation, samples from each mixture were centrifuged for 10 min at 10,000 rpm to separate aqueous and n-octanol phase. The concentration of methylnaltrexone in the aqueous and n-octanol phases was determined by HPLC. Log P was calculated from the ratio of the drug concentration in n-octanol to the drug concentration in water.

### 2. Pharmaceutical Compositions

### 2.1. Capsule—Methylnaltrexone Lauryl Sulfate Ion Pair

| | % (w/w) |
|---|---|
| Methylnaltrexone - Lauryl Sulfate | 20 |
| Labrasol ® | 64 |
| Maisine ® CC | 16 |
| Total | 100 |

Labrasol® and Maisine® CC were mixed according to the proportions listed above. Methylnaltrexone-Lauryl Sulfate was added and the mixture was incubated at about 60° C. in a water bath with mixing for 6 hours until the pharmaceutical composition was obtained as a single phase. Emulsions were prepared by emulsifying 0.5 ml of the pharmaceutical composition in 25 ml of 100 mM phosphate buffer pH 6.8. Mean droplet size and polydispersity index (PDI) were measured via dynamic light scattering. The mean droplet size was about 158 nm, and the PDI was 0.16. The emulsion was also visually examined for any precipitation or phase separation after 12 hours and was found to be stable without any precipitation or phase separation. The pharmaceutical composition was filled in size 00 hard gelatin capsules. Dissolution rate was measured in pH 2 and pH 6.8 media using a USP dissolution apparatus 2 by visual observation of shell dissolution. Capsule shells completely dissolved and released the pharmaceutical composition within 10 mins in both media.

### 2.2. Capsule—Methylnaltrexone Docusate Ion Pair

| | % (w/w) |
|---|---|
| Methylnaltrexone - Docusate | 37 |
| Medium chain triglycerides | 10.71 |
| (MIGLYOL ® 812) | |
| IMWITOR ® 988 | 26.46 |
| Oleic acid | 25.83 |
| Total | 100 |

Medium chain triglycerides, IMWITOR® 988, and oleic acid were mixed according to the proportions listed above. Methylnaltrexone-Docusate was added and incubated at about 60° C. in a water bath with mixing for 6 hours until the pharmaceutical composition was obtained as a single phase. Emulsions were prepared by emulsifying 0.5 ml of the pharmaceutical composition in 25 ml of 100 mM phosphate buffer pH 6.8. Mean droplet size and PDI were measured via dynamic light scattering. The mean droplet size was about 300 nm, and the PDI was 0.45. The emulsion was visually examined for any precipitation or phase separation. The emulsion was stable up to 2 hours, then precipitation was observed.

### 2.3. Enteric Capsule—Methylnaltrexone Docusate Ion Pair

| | % (w/w) |
|---|---|
| Methylnaltrexone - Docusate | 23 |
| Medium chain triglycerides | 13.09 |
| (MIGLYOL ® 812) | |
| IMWITOR ® 988 | 32.34 |
| TWEEN ® 80 | 31.57 |
| Total | 100 |

Medium chain triglycerides, IMWITOR® 988, and TWEEN®80 were mixed according to the proportions listed above. Methylnaltrexone-Docusate was added and incubated at about 60° C. in a water bath with mixing for 12 hours until the pharmaceutical composition was obtained as a single phase. Emulsions were prepared by emulsifying 0.5 ml of the pharmaceutical composition in 25 ml of 100 mM phosphate buffer pH 6.8. Mean droplet size and PDI were measured via dynamic light scattering. The mean droplet size was about 135 nm, and the PDI was 0.27. The emulsion was visually examined for any precipitation or phase separation and was found to be stable without any precipitation or phase separation for up to 12 hours. The pharmaceutical composition was filled in size 0 hard gelatin capsules, which were each then enclosed in size 00 Vcaps® Enteric Capsules. The capsule-in-capsule delivery vehicle was employed, because enteric capsules are not intended for liquid fill and were found to be incompatible with IMWITOR®988. Dissolution rate was measured in pH 2 and pH 6.8 media using a USP dissolution apparatus 2 by visual observation of shell dissolution. Capsule shells did not disintegrate after 2 hours in pH 2 media and completely disintegrated and released the pharmaceutical composition within 7 mins in pH 6.8 media.

### 2.4. Capsule—Methylnaltrexone Docusate Ion Pair

| | % (w/w) |
|---|---|
| Methylnaltrexone - Docusate | 23 |
| Medium chain triglycerides | 13.09 |
| (MIGLYOL ® 812) | |
| IMWITOR ® 988 | 32.34 |
| TWEEN ® 80 | 31.57 |
| Total | 100 |

Medium chain triglycerides, IMWITOR® 988, and TWEEN® 80 were mixed according to the proportions listed above. Methylnaltrexone-Docusate was added and incubated at around 60° C. in a water bath under continuous mixing for 12 hours until the pharmaceutical composition was obtained as a single phase. Emulsions were prepared by emulsifying 0.5 ml of the pharmaceutical composition in 25 ml of 100 mM phosphate buffer pH 6.8. Mean droplet size and PDI were measured via dynamic light scattering. The mean droplet size was about 131 nm, and PDI was 0.16. The emulsion was visually examined for any precipitation or phase separation for 12 hours and was found to be stable without any precipitation or phase separation. The pharmaceutical composition was filled in size 00 hard gelatin capsules. Dissolution rate was measured in pH 2 and pH 6.8 media in USP dissolution apparatus 2 by visual observation of shell dissolution. Capsule shells completely disintegrated and released the pharmaceutical composition within 10 mins in both media.

2.5. Methylnaltrexone Docusate Ion Pair with Varied Drug Loading

The formulations described in Example 2.5 are capsule-based MNTX-DS formulations with varied drug loading and prepared according to the procedures described herein. Formulations 1 and 2 are self-emulsifying (i.e., SEDDS) formulations containing oil, as described herein. Formulations 3 to 6 are micelle-based (i.e., SMDDS) formulations having the same drug loading as formulation 2, which contain surfactants and a cosolvent. As described herein, KOLLIPHOR® RH 40 is a digestible surfactant whereas KOLLIPHOR® HS 15 is a non-digestible surfactant. The goal of preparing these formulations was to observe the outcome of the digestibility of the surfactant on the bioavailability and/or efficacy of MNTX-DS formulations in further animal studies.

Formulation 1: As described hereinbelow, an exemplary formulation includes methylnaltrexone-docusate, IMWITOR® 988, medium chain glyceride (MCT), TWEEN® 80, and a stabilizer (e.g., butylated hydroxytoluene).

| | % (w/w) |
|---|---|
| Methylnaltrexone - Docusate | 10.00 |
| IMWITOR ® 988 | 37.80 |
| Medium Chain Glyceride (MCT) | 13.45 |
| TWEEN ® 80 | 38.70 |
| Butylated Hydroxytoluene (BHT) | 0.05 |
| Total | 100 |

Formulation 2: As described hereinbelow, an exemplary formulation includes methylnaltrexone-docusate, IMWITOR® 988, medium chain glyceride (MCT), TWEEN® 80, and a stabilizer (e.g., butylated hydroxytoluene).

| | % (w/w) |
|---|---|
| Methylnaltrexone - Docusate | 25.00 |
| IMWITOR ® 988 | 31.50 |
| Medium Chain Glyceride (MCT) | 11.20 |
| TWEEN ® 80 | 32.25 |
| Butylated Hydroxytoluene (BHT) | 0.05 |
| Total | 100 |

Formulation 3: As described hereinbelow, an exemplary formulation includes methylnaltrexone-docusate, ethanol, KOLLIPHOR® RH 40, and a stabilizer (e.g., butylated hydroxytoluene).

| | % (w/w) |
|---|---|
| Methylnaltrexone - Docusate | 25.00 |
| Ethanol | 8.33 |
| Vitamin E TPGS | 5.00 |
| KOLLIPHOR ® RH 40 | 61.62 |
| Butylated Hydroxytoluene (BHT) | 0.05 |
| Total | 100 |

Formulation 4: As described hereinbelow, an exemplary formulation includes methylnaltrexone-docusate, ethanol, KOLLIPHOR® RH 40, KOLLIPHOR® HS 15, and a stabilizer (e.g., butylated hydroxytoluene).

| | % (w/w) |
|---|---|
| Methylnaltrexone - Docusate | 25.00 |
| Ethanol | 8.33 |
| Vitamin E TPGS | 5.00 |
| KOLLIPHOR ® RH 40 | 30.81 |
| KOLLIPHOR ® HS 15 | 30.81 |
| Butylated Hydroxytoluene (BHT) | 0.05 |
| Total | 100 |

Formulation 5: As described hereinbelow, an exemplary formulation includes methylnaltrexone-docusate, ethanol, Vitamin E TPGS, and a stabilizer (e.g., butylated hydroxytoluene).

| | % (w/w) |
|---|---|
| Methylnaltrexone - Docusate | 25.00 |
| Ethanol | 8.33 |
| Vitamin E TPGS | 66.62 |
| (as a non-ionic | |
| surfactant) | |
| Butylated Hydroxytoluene (BHT) | 0.05 |
| Total | 100 |

Formulation 6: As described hereinbelow, an exemplary formulation includes methylnaltrexone-docusate, ethanol, GELUCIRE® 44/14, and a stabilizer (e.g., butylated hydroxytoluene).

| | % (w/w) |
|---|---|
| Methylnaltrexone - Docusate | 25.00 |
| Ethanol | 8.33 |
| GELUCIRE ® 44/14 | 66.62 |
| (lauroyl polyoxyl-32 | |
| glycerides NF) | |
| Butylated Hydroxytoluene (BHT) | 0.05 |
| Total | 100 |

### 3. Pharmacokinetics of Liquid Pharmaceutical Compositions in Beagles

In each experiment, a 150 mg dose of methylnaltrexone was administered to each of six dogs, three male and three female. In a first experiment, the 150 mg dose of methylnaltrexone was administered to each dog in a composition according to Example 2.1. In a second experiment, the 150 mg dose of methylnaltrexone was administered to each dog in a composition according to Example 2.2. In a third experiment, the 150 mg dose of methylnaltrexone was administered to each dog in a composition according to Example 2.3. In a fourth experiment, the 150 mg dose of methylnaltrexone was administered to each dog in a composition according to Example 2.4. In a positive control experiment, a RELISTOR® tablet was administered to each dog. Plasma concentrations of methylnaltrexone were measured at 0, 5, 15, 30, 60, 90, 120, 150, 180, 240, 360, 720 minutes post dose.

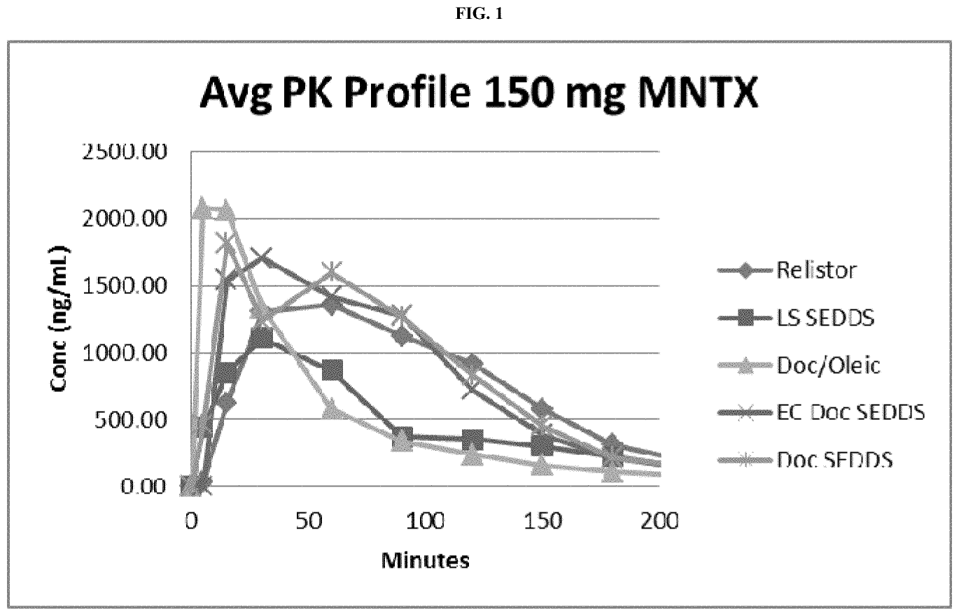

FIG. 1 shows the average plasma concentration of methylnaltrexone after administration of five oral pharmaceutical compositions. The lipid-based formulation comprising methylnaltrexone-docusate (a pharmaceutical composition prepared according to section 2.2) gave rise to the highest average Cₘₐₓ and the shortest average Tₘₐₓ.

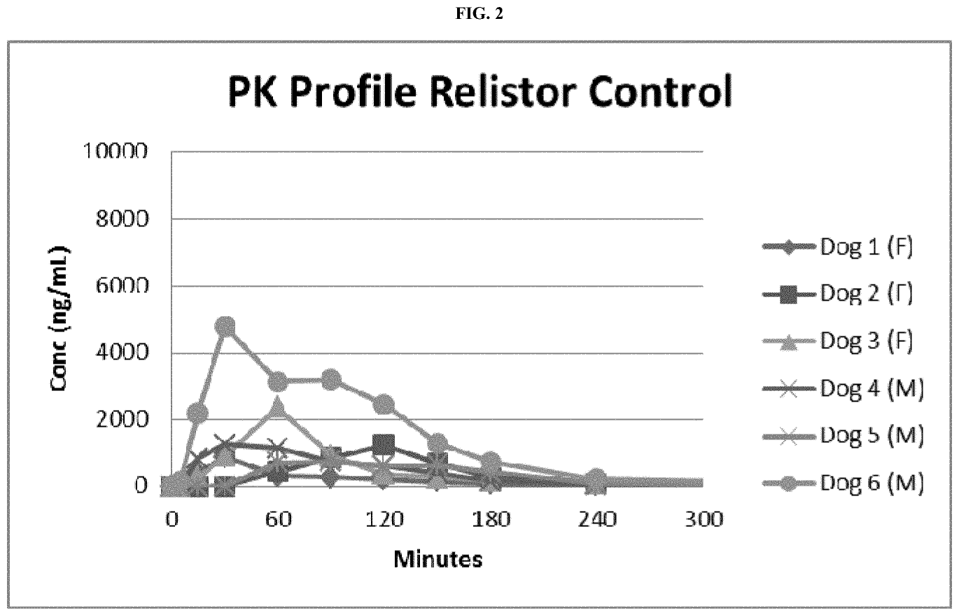

FIG. 2 shows the plasma concentration of methylnaltrexone v. time after administration of a RELISTOR® tablet (control). The maximum plasma concentration was between about 1,000 ng/mL and 5,000 ng/mL. Tₘₐₓ for each of the six dogs was observed within about two hours.

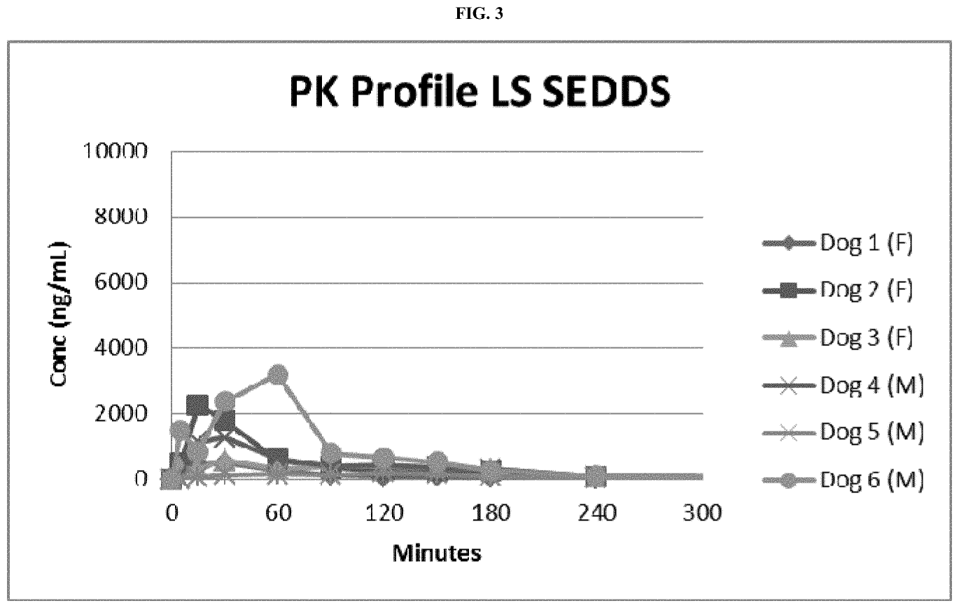

FIG. 3 shows the average plasma concentration of methylnaltrexone v. time after administration of a self-emulsifying drug delivery system comprising methylnaltrexone-lauryl sulfate prepared according to section 2.1 above. The maximum methylnaltrexone concentration was observed at 60 minutes and was less than 4,000 ng/mL.

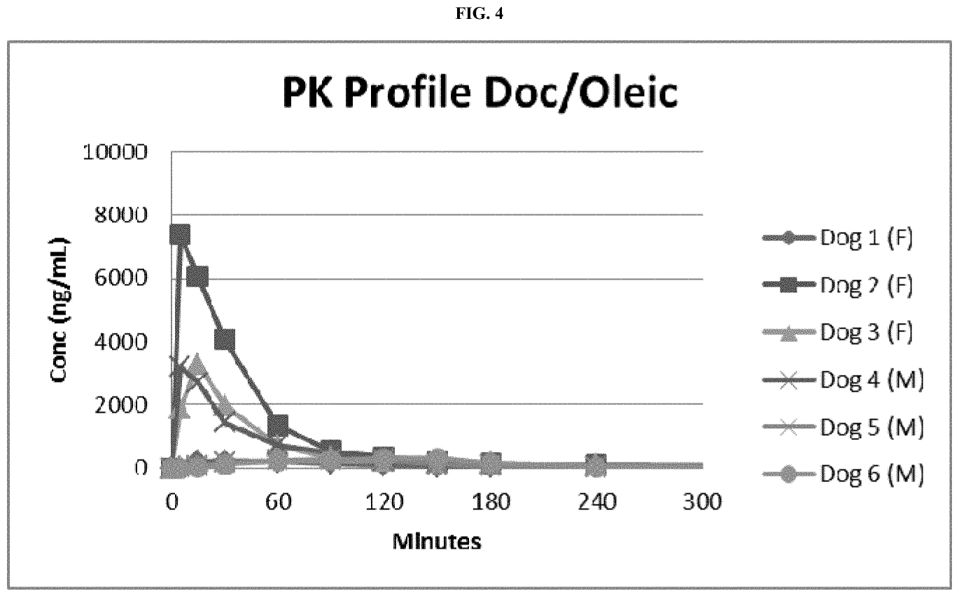

FIG. 4 shows the average plasma concentration of methylnaltrexone v. time after administration of a lipid-based liquid formulation comprising methylnaltrexone-docusate prepared according to section 2.2 above. The peak plasma concentration of methylnaltrexone was observed within 60 minutes for three of the dogs, with a peak plasma concentration of between 6,000 ng/mL and 8,000 ng/mL observed in Dog 2.

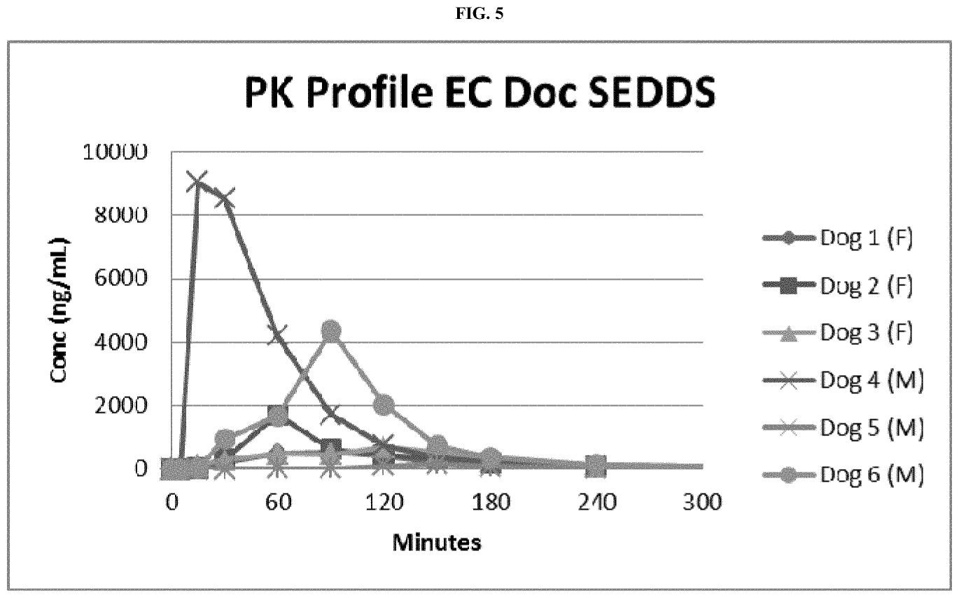

FIG. 5 shows the average plasma concentration of methylnaltrexone v. time after administration of a self-emulsifying drug delivery system comprising methylnaltrexone-docusate in enteric capsules prepared according to section 2.3 above. The maximum plasma concentration of between 8,000 ng/mL and 10,000 ng/mL was observed in Dog 4 within an hour.

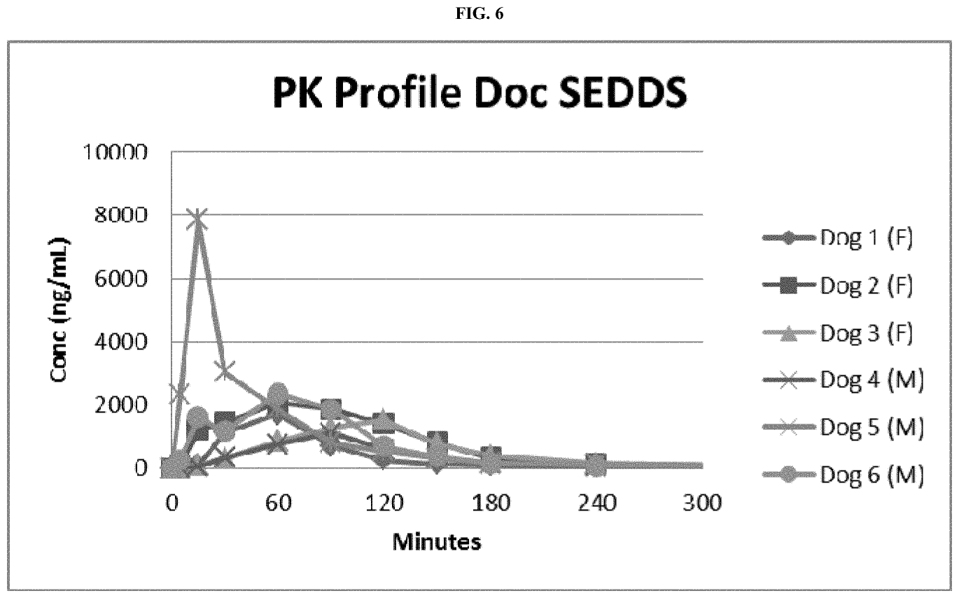

FIG. 6 shows the average plasma concentration of methylnaltrexone v. time after administration of a self-emulsifying drug delivery system comprising methylnaltrexone-docusate prepared according to section 2.4 above. The highest plasma concentration of about 8,000 ng/mL of methylnaltrexone was observed in Dog 5 within an hour of administration.

1. A pharmaceutical composition in a liquid oral dosage form comprising:
(a) an ion pair having the formula: 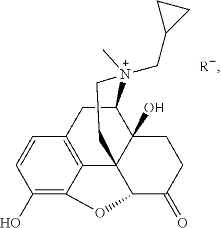
wherein R′ is an anion selected from the group consisting of lauryl sulfate and docusate; and
(b) an oil, surfactant, cosolvent, or combination thereof.

2-3. (canceled)

4. The pharmaceutical composition of claim 1, wherein the pharmaceutical composition comprises about 1% to about 75%, about 10% to about 60%, about 15% to about 50%, or about 20% to about 40% by weight of the ion pair.

5. The pharmaceutical composition of claim 1, (i) wherein the oil comprises at least one oil selected from the group consisting of glyceryl monooleate, glyceryl monolinoleate, propylene glycol dicaprolate/dicaprate, soybean oil, polyglyceryl-3 dioleate, oleic acid, glyceryl caprylate, medium chain triglycerides, and a combination thereof;
(ii) wherein the oil comprises glyceryl monolinoleate;
(iii) wherein the oil comprises oleic acid;
(iv) wherein the oil comprises glyceryl caprylate;
(v) wherein the oil comprises medium chain triglycerides;
(vi) wherein the oil comprises glyceryl caprylate and medium chain triglycerides;
(vii) wherein the pharmaceutical composition comprises at least two oils; and/or
(viii) wherein the total oil content of the pharmaceutical composition is about 10% to about 80%, about 10% to about 20%, about 20% to about 50%, or about 50% to about 70% by weight.

6-13. (canceled)

14. The pharmaceutical composition of claim 1,
(i) wherein the surfactant is selected from the group consisting of oleoyl polyoxyl-6 glycerides, linoleoyl polyoxyl-6 glycerides, caprylocaproyl polyoxyl-8 glycerides, polysorbate 80, polyoxyl 40 hydrogenated castor oil, polyoxyl 15 hydroxystearate, lauroyl polyoxyl-32 glycerides, and a combination thereof;
(ii) wherein the surfactant comprises caprylocaproyl polyoxyl-8 glycerides;
(iii) wherein the surfactant comprises polysorbate 80;
(iv) wherein the surfactant comprises linoleoyl polyoxyl-6 glycerides;
(v) wherein the pharmaceutical composition comprises at least two surfactants; and/or
(vi) wherein the pharmaceutical composition comprises about 10% to about 70%, about 15% to about 40%, or about 20% to about 35% of the surfactant by weight.

15-18. (canceled)

19. The pharmaceutical composition of claim 1, wherein the pharmaceutical composition comprises about 1 mg to about 100 mg, about 50 mg to about 900 mg, about 75 mg to about 850 mg, about 100 mg to about 850 mg, about 150 mg to about 850 mg, about 200 mg to about 800 mg, or about 200 mg to about 700 mg of the ion pair, and/or at least about 1 mg, about 10 mg, about 25 mg, about 50 mg, about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg or about 900 mg of the ion pair.

20. (canceled)

21. The pharmaceutical composition of claim 14 further comprising water, wherein the liquid composition comprises an emulsion, optionally which forms upon contact with aqueous liquids.

22. (canceled)

23. A pharmaceutical composition in a liquid oral dosage form comprising:
(a) an ion pair having the formula: 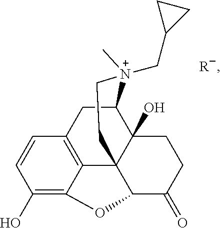
wherein R′ is an anion selected from the group consisting of lauryl sulfate and docusate.

24-25. (canceled)

26. A pharmaceutical composition in a liquid oral dosage form comprising (i) methylnaltrexone, (ii) lauryl sulfate or docusate and (iii) one or more of an oil, surfactant, and a cosolvent, wherein the methylnaltrexone and lauryl sulfate or docusate are present in substantially equal molar amounts.

27-28. (canceled)

29. The pharmaceutical composition of claim 26, wherein the pharmaceutical composition comprises about 1% to about 75%, about 10% to about 60%, about 15% to about 50%, or about 20% to about 40% by weight of the methylnaltrexone and lauryl sulfate or docusate.

30. (canceled)

31. The pharmaceutical composition of claim 26,
(i) wherein the oil comprises at least one of glyceryl monooleate, glyceryl monolinoleate, propylene glycol dicaprolate/dicaprate, soybean oil, polyglyceryl-3 dioleate, oleic acid, glyceryl caprylate, medium chain triglycerides, and a combination thereof;
(ii) wherein the oil comprises glyceryl monolinoleate;
(iii) wherein the oil comprises oleic acid;
(iv) wherein the oil comprises glyceryl caprylate;
(v) wherein the oil comprises medium chain triglycerides;
(vi) wherein the composition comprises a combination of glyceryl caprylate and medium chain triglycerides;
(vii) wherein the composition comprises at least two oils; and/or
(viii) wherein the total oil content of the pharmaceutical composition is about 10% to about 80%, about 10% to about 20%, about 20% to about 50%, or about 50% to about 70% by weight.

32-39. (canceled)

40. The pharmaceutical composition of claim 26,
(i) wherein the surfactant is selected from the group consisting of oleoyl polyoxyl-6 glycerides, linoleoyl polyoxyl-6 glycerides, caprylocaproyl polyoxyl-8 glycerides, polysorbate 80, polyoxyl 40 hydrogenated castor oil, polyoxyl 15 hydroxystearate, lauroyl polyoxyl-32 glycerides, and a combination thereof;
(ii) wherein the surfactant comprises caprylocaproyl polyoxyl-8 glycerides;
(iii) wherein the surfactant comprises polysorbate 80;
(iv) wherein the surfactant comprises linoleoyl polyoxyl-6 glycerides;
(v) wherein the pharmaceutical composition comprises at least two surfactants; and/or
(vi) wherein the pharmaceutical composition comprises about 10% to about 70%, about 15% to about 40%, or about 20% to about 35% of the surfactant by weight.

41-44. (canceled)

45. The pharmaceutical composition of claim 26, wherein the pharmaceutical composition comprises
(i) about 1 mg to about 100 mg, about 50 mg to about 800 mg, about 100 mg to about 750 mg, about 150 mg to about 750 mg, or about 200 mg to about 700 mg of the methylnaltrexone and lauryl sulfate;
(ii) at least about 1 mg, about 10 mg, about 25 mg, about 50 mg, about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, or about 700 mg of the methylnaltrexone and lauryl sulfate;
(iii) about 1 mg to about 100 mg, about 50 mg to about 900 mg, about 100 mg to about 900 mg, about 150 mg to about 850 mg, or about 200 mg to about 800 mg; or
(iv) at least about 1 mg, about 10 mg, about 25 mg, about 50 mg, about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg about 700 mg, about 750 mg, about 800 mg, about 850 mg, or about 900 mg of the methylnaltrexone and docusate.

46-48. (canceled)

49. The pharmaceutical composition of claim 26, further comprising water, wherein the liquid composition comprises an emulsion, optionally which forms upon contact with aqueous liquids.

50. (canceled)

51. The pharmaceutical composition of claim 1, comprising a surfactant and a cosolvent, optionally, wherein the cosolvent is selected from the group consisting of triacetin, ethanol, glycerol, propylene glycol, and polyethylene glycol.

52. (canceled)

53. The pharmaceutical composition of claim 1, further comprising a stabilizer, optionally, wherein the stabilizer is (i) selected from the group consisting of butylated hydroxytoluene (BHT), butylated hydroxyanisole (BHA), propyl gallate, ascorbic acid-6-palmitate, alpha tocopherol, Vitamin E TPGS and a combination thereof, and/or (ii) is present in an amount of at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10% by weight of the composition.

54-55. (canceled)

56. A salt having the formula 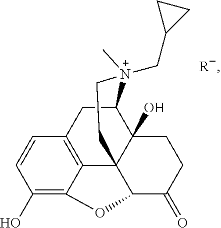
wherein R′ is docusate.

57. A pharmaceutical composition comprising the salt of claim 56, optionally wherein the composition is a liquid composition for oral administration.

58. (canceled)

59. The pharmaceutical composition of claim 57, further comprising at least one of an oil, a surfactant, or a cosolvent.

60. (canceled)

61. The pharmaceutical composition of claim 1, wherein the composition is in a capsule.

62. A method of treating opioid induced constipation in a subject in need thereof comprising orally administering the pharmaceutical composition of claim 1 to the subject.

63. The method of claim 62, wherein oral administration of the pharmaceutical composition to the subject results in (i) a Cₘₐₓ of methylnaltrexone ranging from about 50 ng/mL to about 200 ng/mL; and/or (ii) a Tₘₐₓ of methylnaltrexone that is less than about 4 hours, less than about 2 hours, less than about 1 hour, less than about 30 minutes, less than about 15 minutes, or less than about 10 minutes.

64. (canceled)
